(19) United States
(12) Patent Application Publication
(10) Pub. No.: US 2005/0192289 A1
(43) Pub. Date: Sep. 1, 2005

(54) Novel cinnamic amides

(75) Inventors: Eric Wellner, Lund (SE); Helena Sandin, Lund (SE)

Correspondence Address: BROWDY AND NEIMARK, P.L.L.C.;624 NINTH STREET, NW, SUITE 300, WASHINGTON, DC, 20001-5303, US

(73) Assignee: Active Biotech AB, Lund (SE)

(21) Appl. No.: 10/995,036

(22) Filed: Nov. 23, 2004

(30) Foreign Application Priority Data
Feb. 25, 2004 (SE) 0400440-4

(51) Int. Cl. A61K 31/495

(52) U.S. Cl. 514/255.01; 544/386

(57) ABSTRACT

E-cinnamic amides of piperazine derivatives according to formula (I) 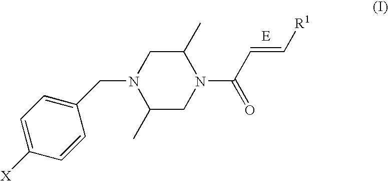
wherein X is chloro or fluoro and R¹ is an aromatic or heteroaromatic group, their pharmaceutically acceptable salts or solvates. The invention also relates to pharmaceutical compositions containing a compound of formula (I) together with a pharmaceutically acceptable carrier. Included are also processes for the preparation of compounds of formula (I), as well as methods for treating mammals suffering from inflammatory, autoimmune, proliferative or hyperproliferative diseases by administering a compound having the formula (I) to said mammal.

### FIELD OF THE INVENTION

This invention relates to novel E-cinnamic amides of trans-2,5-dimethyl-piperazine derivatives, their pharmaceutically acceptable salts, pharmaceutical compositions containing them and their use in therapy.

Another aspect of the invention is a method of treating inflammatory, autoimmune, proliferative and hyperproliferative diseases. A preferred method is the method of treating rheumatoid arthritis, atherosclerosis, systemic sclerosis, multiple sclerosis, Alzheimer's disease, encephalomyelitis, systemic lupus erythematosus, Guillian-Barre syndrome, allograft rejection, urticaria, angioderma, allergic conjunctivitis, atopic dermatitis, allergic contact dermatitis, drug or insect sting allergy, systemic anaphylaxis, proctitis, inflammatory bowel disease or asthma.

### BACKGROUND

Chemokines are small secreted cytokines consisting of 8-14 kDa proteins, which can be classified into four groups according to the sequence of their conserved cysteine residues, CXC, CC, C and CX₃C. They promote upregulation of cellular adhesion molecules, which enforces adhesion and lead to cell migration. Hence, the chemotactic cytokines play a crucial part in the recruitment and trafficking of leukocyte subsets.

Among the CC chemokines, MIP-1α and RANTES, known as ligands for CCR1, CCR3, CCR4 and CCR5 receptors, are involved in autoimmune diseases such as rheumatoid arthritis, inflammatory bowel disease and multiple sclerosis. This is strongly supported by the fact that CCR1 knockout mice show a significantly reduced incidence of disease in a mouse EAE model compared with the wild type mice. Studies by Karpus et al. (J. Immunol. 1995, 155, 5003) further prove the pivotal role of MIP-1α in the same model of multiple sclerosis. It was shown that antibodies to MIP-1α prevented the development of both acute and relapsing paralytic disease as well as infiltration of mononuclear cells into the CNS.

In addition, there is strong evidence implicating RANTES in the pathophysiology of rheumatoid arthritits. For example, RANTES mRNA was detected in synovial tissue samples from patients with rheumatoid arthritis (Snowden, N. et al., Lancet, 1994, 343, 547). Further, antibodies to RANTES greatly reduced the development of disease in an adjuvant-induced arthritis model in the rat.

A number of studies have provided evidence for a role of CCR1 in allograft rejection. Combining a sub-nephrotoxic amount of cyclosporin A with blockade of chemokine receptors using a CCR1 antagonist has been shown to have a positive effect on solid allograft survival (Horuk, R. et al., J. Biol. Chem. 2001, 276, 4199).

Therefore, molecules that inhibit the interaction between the inflammatory chemokines and their receptor would be beneficial in the treatment of inflammatory, autoimmune, proliferative and hyperproliferative diseases.

### RELATED DISCLOSURES

The U.S. Pat. No. 4,368,199 discloses piperazinyl substituted cinnamic amides as being useful in the treatment of heart diseases. The focus of this patent application lies on 3,4,5-trimethoxycinnamoylpiperazine derivatives, which are N-substituted with variously arylated alkyl-spacers. The most common spacer length consists of two C-units

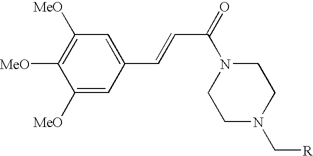

The international patent application WO 98/56771 claims benzylated piperazines useful in the treatment of inflammatory disorders by inhibition of the activity of chemokines. Examples of the most preferred compounds are summarised in Table 1.

TABLE 1

| Structure | X | Y | Z |
|---|---|---|---|
| 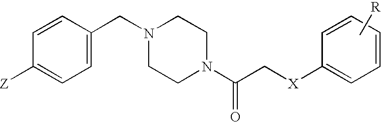 | O | | F, Cl |
| 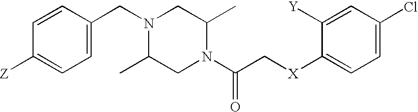 | O N CH′, R′ ≠ H | glycinamido, ureido, aminocarbonyl aminocarbonyl, ureido, glycinamido, H aminocarbonyl, ureido, glycinamido, H | F |

Footnote:
All 2,5-dimethylpiperazine derivatives have been synthesized and tested as racemic mixtures.

One benzylcinnamoyl-piperazine derivative, 1-(4-chlorobenzyl)-4-(2,4-dichlorocinnamoyl)-piperazine, is published in the U.S. Pat. No. 4,742,062 in the synthesis of remedies against hyperlipidemia.

The U.S. Pat. No. 4,616,086 discloses 1-cinnamoyl-piperazine-4-yl-methylbenzoic acid derivatives and esters thereof as drugs against hyperlipidemia.

Caignard et al. (Eur. J. Med. Chem. 2000, 35, 107) publishes certain cinnamic amides of benzylpiperazine with low affinity to the σ-site.

### DESCRIPTION OF THE INVENTION

It has now surprisingly been found that the compounds of formula (I) 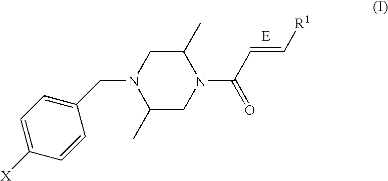
wherein
the double bond in the amide moiety of formula (I) has an E-configuration;
X is a fluorine or a chlorine atom;
the methyl groups located at the 2- and 5-position of the piperazine ring are in trans-configuration to each other;
R¹ represents:
   a) an aromatic group represented by the formula: 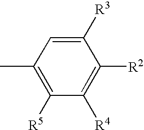
      wherein:
R² is a substituent with a π-value between 0.5 and 0.9 and a molrefractory-value (MR) between 5.0 and 9.0 such as methyl, chloro, bromo, trifluoromethyl, or R² is a nitro or methoxy substituent;
R³ is selected from hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from hydrogen and methoxy, with the provisos that if R² is methoxy, R⁴ is selected from a group consisting of hydrogen, chloro, bromo or methoxy, or, if R³ is hydrogen, R⁴ is hydrogen;
R⁵ is hydrogen, chloro, methyl, with the proviso that if R⁵ is chloro or methyl, X is fluoro, R² is chloro or methyl and R³ is hydrogen;
b) a heteroaromatic group represented by the formula: 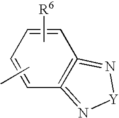
   wherein:
Y is O or S;
R⁶ is one or more substituents independently selected from hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
c) a heteroaromatic group represented by the formula: 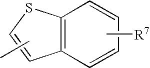
   wherein:
R⁷ is one or more substituents independently selected from hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
or a pharmaceutically acceptable salt or solvate thereof;
   are unexpectedly effective in inhibiting the signalling of the chemokine receptor CCR1.

A preferred group of compounds of formula (I) is that group of compounds wherein: R¹ represents:
a) an aromatic group represented by the formula: 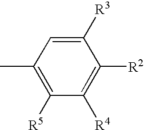
   wherein:
R² is selected from methyl, chloro, bromo, trifluoromethyl, nitro and methoxy;
R³ is selected from hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from hydrogen and methoxy, with the provisos that if R² is methoxy, R⁴ is selected from a group consisting of hydrogen, chloro, bromo or methoxy, or, if R³ is hydrogen, R⁴ is hydrogen;
R⁵ is hydrogen, chloro, methyl, with the proviso that if R⁵ is chloro or methyl, X is fluoro, R² is chloro or methyl and R³ is hydrogen;
b) a heteroaromatic group represented by the formula: 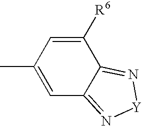
   wherein:
R⁶ is one or more substituents independently selected from hydrogen, halo, methyl, ethyl, haloalkyl, alkoxy, haloalkoxy and nitro;
c) a heteroaromatic group represented by the formula: 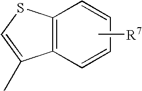
   wherein:
R⁷ is one or more substituents independently selected from hydrogen, halo, C1-C3 alkyl, haloalkyl, alkoxy, haloalkoxy, nitro, cyano, alkylamlino, aryl, alkylcarbonyl, aminocarbonyl;
   or a pharmaceutically acceptable salt or solvate thereof.

Preferred compounds are:
(E)-(trans)-3-(4-Bromo-phenyl)-1-[4-(4-fluoro-benzyl)-2,5-dimethyl-piperazine-1-yl]-prop-2-en-1-one
(E)-(trans)-3-(4-Chloro-3-nitro-phenyl)-1-[4-(4-chloro-benzyl)-2,5-dimethyl-piperazine 1-yl]-prop-2-en-1-one
(E)-(trans)-3-(3,4-Dichloro-phenyl)-1-[4-(4-fluoro-benzyl)-2,5-dimethyl-piperazine-1-yl]-prop-2-en-1-one
(E)-(trans)-1-[4-(4-Fluoro-benzyl)-2,5-dimethyl-piperazine-1-yl]-3-p-tolyl-prop-2-en-1-one
(E)-(trans)-1-[4-(4-Fluoro-benzyl)-2,5-dimethyl-piperazine-1-yl]-3-(4-nitro-phenyl)-prop-2-en-1-one
(E)-(trans)-3-(2,4-Dichloro-phenyl)-1-[4-(4-fluoro-benzyl)-2,5-dimethyl-piperazine-1-yl]-prop-2-en-1-one hydrochloride
(E)-(trans)-3-Benzo[b]thiophen-3-yl-1-[4-(4-fluoro-benzyl)-2,5-dimethyl-piperazine 1-yl]-prop-2-en-1-one
(E)-(trans)-3-(3,4-Dichloro-phenyl)-1-[4-(4-chloro-benzyl)-2,5-dimethyl-piperazine-1-yl]-prop-2-en-1-one
(E)-(trans)-3-(3,4-Dimethoxy-phenyl)-1-[4-(4-fluoro-benzyl)-2,5-dimethyl-piperazine-1-yl]-prop-2-en-1-one
(E)-(trans)-3-(3-Bromo-4,5-dimethoxy-phenyl)-1-[4-(4-fluoro-benzyl)-2,5-dimethyl-piperazine-1-yl)-prop-2-en-1-one
(E)-(trans)-3-(4-Chloro-3-trifluoromethyl-phenyl)-1-[4-(4-fluoro-benzyl)-2,5-dimethyl-piperazine-1-yl]-prop-2-en-1-one
(E)-(trans)-3-Benzo[2,1,3]oxadiazol-5-yl-1-[4-(4-fluoro-benzyl)-2,5-dimethyl-piperazine-1-yl]-prop-2-en-1-one
(E)-(trans)-3-(2,4-Dimethyl-phenyl)-1-[4-(4-fluoro-benzyl)-2,5-dimethyl-piperazine-1-yl]-prop-2-en-1-one
(E)-(trans)-1-[4-(4-Chloro-benzyl)-2,5-dimethyl-piperazine-1-yl]-3-(4-chloro-phenyl)-prop-2-en-1-one
(E)-(trans)-1-[4-(4-Fluoro-benzyl)-2,5-dimethyl-piperazine-1-yl]-3-(4-methyl-3-nitro-phenyl)-prop-2-en-1-one
(E)-(trans)-1-[4-(4-Chloro-benzyl)-2,5-din ethyl-piperazine-1-yl]-3-(4-methyl-3-nitro-phenyl)-prop-2-en-1-one
(E)-(trans)-3-Benzo[2,1,3]thiadiazol-5-yl-1-[4-(4-fluoro-benzyl)-2,5-dimethyl-piperazine-1-yl]-prop-2-en-1-one
(E)-(trans)-1-[4-(4-Fluoro-benzyl)-2,5-dimethyl-piperazine-1-yl]-3-(3,4,5-trimethoxy-phenyl)-prop-2-en-1-one
(E)-(trans)-3-(3-Chloro-4-nitro-phenyl)-1-[4-(4-fluoro-benzyl)-2,5-dimethyl-piperazine-1-yl]-prop-2-en-1-one
(E)-(trans)-3-(4-Chloro-3-methoxy-5-nitro-phenyl)-1-[4-(4-fluoro-benzyl)-2,5-dimethyl-piperazine-1-yl]-prop-2-en-1-one
(E)-(trans)-1-[4-(4-Fluoro-benzyl)-2,5-dimethyl-piperazine-1-yl]-3-(4-trifluoromethyl-phenyl)-prop-2-en-1-one
(E)-(trans)-1-[4-(4-Fluoro-benzyl)-2,5-dimethyl-piperazine-1-yl]-3-(3-trifluoromethyl-4-nitro-phenyl)-prop-2-en-1-one
(E)-(trans)-3-(4-Chloro-3-methoxy-phenyl)-1-[4-(4-fluoro-benzyl)-2,5-dimethyl-piperazine-1-yl]-prop-2-en-1-one
(E)-(trans)-3-(3-Chloro-4,5-dimethoxy-phenyl)-1-[4-(4-fluorobenzyl)-2,5-dimethyl-piperazine-1-yl]-prop-2-en-1-one
(E)-(trans)-3-(4-Bromo-3,5-dimethoxy-phenyl)-1-[4-(4-fluorobenzyl)-2,5-dimethyl-piperazine-1-yl]-prop-2-en-1-one
(E)-(trans)-1-[4-(4-Fluorobenzyl)-2,5-dimethyl-piperazine-1-yl)-3-(3-methoxy-4-methyl-phenyl)-prop-2-en-1-one
(E)-(trans)-3-(4-Bromo-benzo[2,1,3]oxadiazol-6-yl)-1-[4-(4-fluorobenzyl)-2,5-dimethyl-piperazine-1-yl]-prop-2-en-1-one
(E)-(trans)-3-(4-Bromo-phenyl)-1-[4-(4-chlorobenzyl)-2,5-dimethyl-piperazine-1-yl]-prop-2-en-1-one
(E)-(trans)-1-[4-(4-Chlorobenzyl)-2,5-dimethyl-piperazine-1-yl]-3-(4-nitro-phenyl)-prop-2-en-1-one
(E)-(trans)-3-(3-Bromo-4-chloro-phenyl)-1-[4-(4-fluorobenzyl)-2,5-dimethyl-piperazine-1-yl)-prop-2-en-1-one
(E)-(trans)-3-(4-Bromo-3-chloro-phenyl)-1-[4-(4-fluorobenzyl)-2,5-dimethyl-piperazine-1-yl]-prop-2-en-1-one
(E)-(trans)-3-(3,4-Dibromo-phenyl)-1-[4-(4-fluorobenzyl)-2,5-dimethyl-piperazine-1-yl]-prop-2-en-1-one
(E)-(trans)-3-(4-Bromo-3-nitro-phenyl)-1-[4-(4-fluorobenzyl)-2,5-dimethyl-piperazine-1-yl]-prop-2-en-1-one
(E)-(trans)-3-(4-Chloro-benzo[2,1,3]oxadiazol-6-yl)-1-[4-(4-fluorobenzyl)-2,5-dimethyl-piperazine-1-yl]-prop-2-en-1-one
(E)-(trans)-3-(4-Bromo-benzo[2,1,3]thiadiazol-6-yl)-1-[4-(4-fluorobenzyl)-2,5-dimethyl-piperazine-1-yl]-prop-2-en-1-one
(E)-(trans)-3-(4-Chloro-benzo[2,1,3]thiadiazol-6-yl) 1-[4-(4-fluorobenzyl)-2,5-dimethyl-piperazine-1-yl]-prop-2-en-1-one
(E)-(trans)-3-(4-Bromo-5-methoxy-benzo[2,1,3]thiadiazol-6-yl)-1-[4-(4-fluorobenzyl)-2,5-dimethyl-piperazine-1-yl]-prop-2-en-1-one
(E)-(trans)-1-[4-(4-Fluoro-benzyl)-2,5-dimethyl-piperazine-1-yl]-3-(4-nitro-benzo[2,1,3]thiadiazol-5-yl)-prop-2-en-1-one
(E)-(trans)-3-(4-Chloro-phenyl)-1-[4-(4-fluoro-benzyl)-2,5-dimethyl-piperazine-1-yl]-prop-2-en-1-one
(E)-(trans)-3-(4-Chloro-3-nitro-phenyl)-1-[4-(4-fluoro-benzyl)-2,5-dimethyl-piperazine-1-yl]-prop-2-en-1-one

Examples of the preferred compounds of the invention in the above formula (I) have substituents as shown in the following Table 2.

TABLE 2

| Compound No. | X | R¹ |
|---|---|---|
| 6.1 | F | 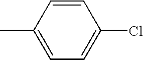 |
| 5.3 | F | 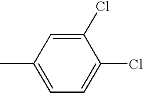 |
| 5.6 | F | 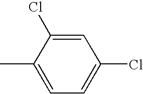 |
| 6.2 | F | 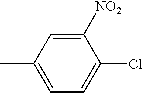 |
| 5.39 | F | 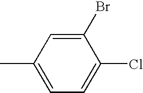 |
| 5.12 | F | 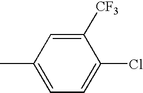 |
| 5.26 | F | 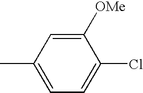 |
| 5.22 | F | 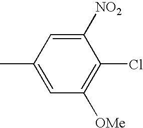 |
| 5.1 | F | 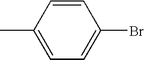 |
| 5.42 | F | 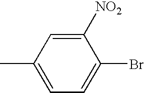 |
| 5.40 | F | 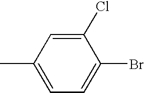 |
| 5.41 | F | 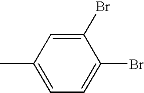 |
| 5.29 | F | 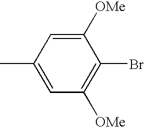 |
| 5.23 | F | 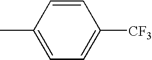 |
| 5.4 | F | 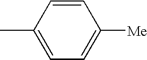 |
| 5.14 | F | 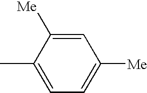 |
| 5.16 | F | 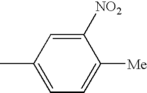 |
| 5.30 | F | 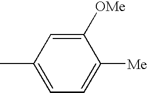 |
| 5.10 | F | 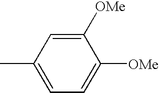 |
| 5.27 | F | 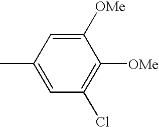 |
| 5.11 | F | 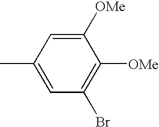 |
| 5.19 | F | 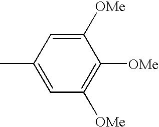 |
| 5.5 | F | 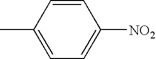 |
| 5.21 | F | 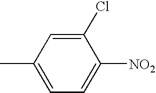 |
| 5.25 | F | 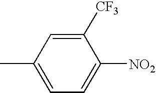 |
| 5.8 | F | 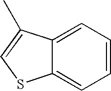 |
| 5.13 | F | 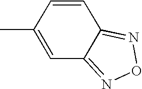 |
| 5.43 | F | 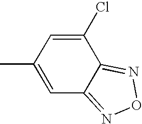 |
| 5.32 | F | 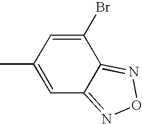 |
| 5.18 | F | 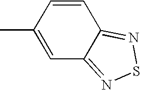 |
| 5.46 | F | 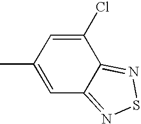 |
| 5.45 | F | 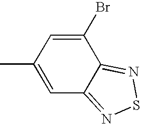 |
| 5.47 | F | 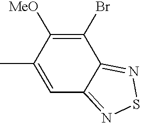 |
| 5.48 | F | 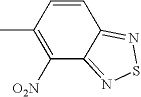 |
| 5.15 | Cl | 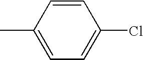 |
| 5.2 | Cl | 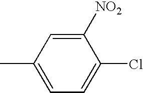 |
| 5.9 | Cl | 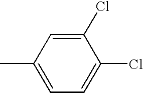 |
| 5.33 | Cl | 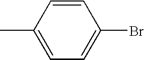 |
| 5.17 | Cl | 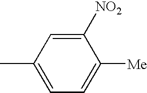 |
| 5.36 | Cl | 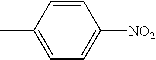 |

Definitions

The term “therapy” and “treatment” as used herein includes prophylaxis as well as relieving the symptoms of disease.

In the context of the present specification, an alkyl, alkenyl or alkynyl substituent group or all alkyl, alkenyl or alkynyl moiety in a substituent group may be a branched or straight chain or cyclic. Further, a nitrogen atom may be monosubstituted or independently disubstituted with the same or different alkyl, alkenyl or alkynyl moieties.

Unless specified otherwise,

“Alkyl” refers to a hydrocarbon group joined by single carbon-carbon bonds and having 1-4 carbon atoms, selected from methyl, ethyl, n-propyl, isopropyl, cyclopropyl, n-butyl, isobutyl, sec-butyl, tert-butyl and cyclobutyl. The descriptors C-1 to C-4 refer to the number of carbon atoms present in the alkyl group.

“Alkenyl” refers to a hydrocarbon group comprising one double carbon-carbon bond and having 2-4 carbon atoms.

“Alkynyl” refers to a hydrocarbon group comprising one triple carbon-carbon bond and having 2-4 carbon atoms.

“Alkoxy” refers to the radical —OR_{Alk} wherein R_{Alk} is alkyl as defined above.

“Halo” or “halogen” refers to fluoro, chloro, bromo or iodo.

“Haloalkyl” refers to an alkyl radical as defined above, that is substituted by one or more halo radicals, e.g., trifluoromethyl, difluoromethyl, pentafluoroethyl, trichloromethyl and the like.

“Haloalkoxy” refers to radical or the formula —OR_{Hal} where R_{Hal} is a haloalkyl radical as defined above.

“Nitro” refers to the radical —NO₂.

“Carboxy” refers to the radical —C(O)OH or —C(O)O⁻.

“Cyano” refers to the radical —CN.

CHCl₃ refers to chloroform.

CH₂Cl₂ refers to dichloromethane.

“Hydroxy” refers to a radical —OH.

“Hydroxyalkyl” refers to an alkyl radical as defined above substituted by a hydroxy radical.

“Alkylthio” refers to a radical of the formula —S—R_{Alk} where R_{Alk} is an alkyl radical as defined above.

“Alkylsulfonyl” refers to a radical of the formula —S(O)₂R_{Alk} where R_{Alk} is an alkyl radical as defined above.

“Alkylsulfinyl” refers to a radical of the formula —S(O)R_{Alk} where R_{Alk} is an alkyl radical as defined above.

“Amino” refers to a radical of the formula —NH₂.

“Alkyl amino” refers to a radical of the formula —N(H)R_{Alk} where R_{Alk} is an alkyl radical as defined above; or —N(R_{Alk})₂ wherein R_{Alk} independently represents the same or different alkyl radicals as defined above.

“Aryl” refers to an optionally substituted aromatic group with at least one ring having a conjugated π-electron system, containing up to two conjugated and/or fused ring systems. Aryl includes carbocyclic aryl and biaryl groups, all of which may be optionally substituted Substituents are selected from halogen, C1-C4 alkyl, NH₂, OCF₃, CF₃, alkoxy, alkylthio, CN, alkylsulfonyl and NO₂, as defined above.

“Alkylsulfonylamino” refers to a radical of the formula —N(H)—S(O)₂R_{Alk} where R_{Alk} is an alkyl radical as defined above.

“Sulfonamido” refers to a radical of the formula —S(O)₂NH₂.

“Dialkylsulfonamido” refers to a radical of the formula —S(O)₂N(R_{Alk})₂ wherein R_{Alk} independently represents the same or different alkyl radicals as defined above.

“Alkylcarbonyl” refers to a radical of the formula —C(O)R_{Alk} where R_{Alk} is an alkyl radical as defined above.

“Alkoxycarbonylalkyl” refers to a radical of the formula —C(O)OR_{Alk} where R_{Alk} is an alkyl radical as defined above.

“Aminocarbonyl” refers to a radical of the formula —C(O)N₂.

“Alkylaminocarbonyl” refers to a radical of the formula —C(O)N(H)R_{Alk} where R_{Alk} is an alkyl radical as defined above; or to a radical of the formula —C(O)N(R_{Alk})₂ wherein R_{Alk} independently represents the same or different alkyl radicals as defined above.

“Ureido” is a radical of the formula —N(H)C(O)NH₂.

“Heteroaryl” refers to an optionally substituted aromatic group with at least one ring having a conjugated π-electron system, containing up to two conjugated and/or fused ring systems and 1-3 heteroatoms selected from O, S and N. Heteroaryl includes carbocyclic heteroaryl, aryl-heteroaryl and biheteroaryl groups, all of which may be optionally substituted. Substituents are selected from halogen, C1-C4 alkyl, NH₂, OCF₃, CF₃, alkoxy, alkylthio, CN, alkylsulfonyl and NO₂, as defined above. Examples of heteroaryl rings include pyrrole, furan, thiophene, indole, isoindole, benzofuran, isobenzofuran, benzothiophene, pyridine, quinoline, isoquinoline, quinolizine, pyrazole, imidazole, isoxazole, oxazole, isothiazole, thiazole, pyridazine, pyrimidine, and pyrazine.

The descriptor “trans” indicates that the two methyl groups are located on opposite sides of the piperazine plane. The descriptor “cis” indicates that the two methyl groups are located at the same side of the piperazine plane. The descriptor “E” indicates that the substituents on the double bond of the amide moiety are “entgegen” meaning opposite.

Description and values of the pi and MR parameters can be found in Hansch, C., and Leo, A., Exploring QSAR: Fundamentals and Applications in Chemistry and Biology. ACS, Washington, D.C. 1995 and Hansch, C., Leo, A, and Hoekman, D., Exploring QSAR: Hydrophobic, Electronic, and Steric Constants. ACS, Washington, D.C. 1995.

Structure Activity Relationship

Prior Art Compounds

1-[4-(4-Fluoro-benzyl)-piperazine-1-yl]-3-(3,4,5-trimethoxy-phenyl)-prop-2-en-1-one, 3-(4-chloro-phenyl)-1-[4-(4-fluoro-benzyl)-piperazine-1-yl]-prop-2-en-1-one and 1-(4-chlorobenzyl)-4-(2,4-dichlorocinnamoyl)-piperazine are included as prior art compounds hereinafter called Compound A, B and C respectively. Compound A and B are described in the international patent application WO 98/56771 (page 118, lines 25 and 19, respectively). The E-isomer of Compound A is described and claimed in the U.S. Pat. No. 4,368,199. Compound C is described in the U.S. Pat. No. 4,742,062.

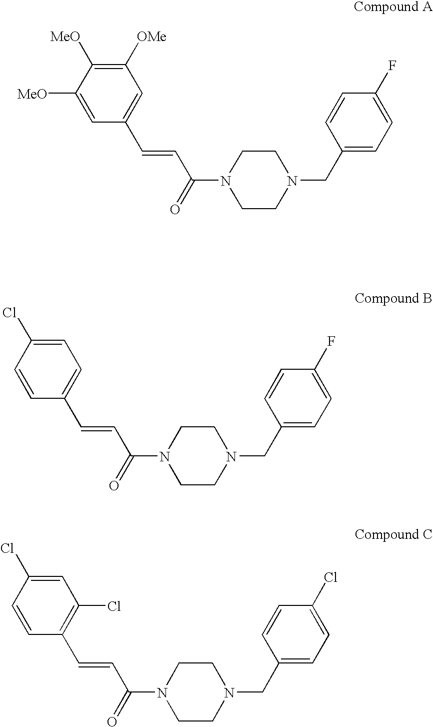

Compared to the prior art Compounds A, B (E- and Z-configurations) and C and various reference compounds, the compounds of the invention showed an increased affinity for the CCR1 receptor in the affinity binding assay (see Table 3 below). Further, they were surprisingly stronger inhibitors in the Ca²⁺-flux assay than the prior art and reference compounds. The improved potency of the compounds, where R¹ is an aromatic group, correlates amongst others to three structural features, viz:
1. The introduction of X (chloro or preferably fluoro) in p-position of the benzylpiperazine moiety increases potency and affinity significantly. There is no teaching of this effect within the prior art. However, the replacement of X with another functional group, e.g., alkyl, or hydrogen decreases the potency and the affinity.
2. The two methyl groups in 2,5-position in formula (I) are in trans-configuration. The replacement of the methyl groups in trans-2,5-position by a substitution e.g. in 2,6-, in 3,5-position or with hydrogen as well as changing the orientation to a cis-2.5 substitution, dramatically decreases the potency of the compound in the Ca²⁺-flux assay and the affinity-binding assay.
3. The configuration of the double bond in the cinnamic amide moiety is E. Changing the configuration from E- to Z-configuration of the double bond in the cinnamic amide part decreases the potency as well as the affinity. Reduction of the double bond to an ethylene group or a substitution of the double bond decreases both potency and affinity.

The invention, combining the features according to 1, 2 and 3 above, provides compounds having a surprising and unexpected potency and affinity.

Furthermore, the oral bioavailability is the fraction of dose absorbed via oral administration and describes the rate and amount of the compounds of the invention reaching the systemic circulation. It is therefore crucial to optimise the bioavailability to improve the pharmacokinetic aspects of compounds.

Preparation of Compounds

The present invention further provides a process for the preparation of a compound of formula (I) by any of the methods given below

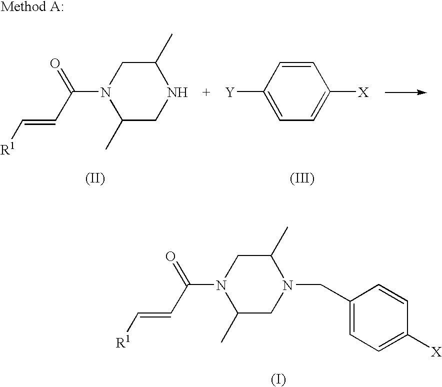

The compounds of formula (I) may be prepared by known methods, for example, as shown above by reaction of a piperazine derivative of formula (II) with a benzaldehyde of formula (III) wherein X is defined in formula (I) and Y is a formyl group (—CHO). This type of reductive amination is known from literature e.g., in Berger et al., Bioorg. Med. Chem. Lett. 2002, 12, 2989. Another example is the reaction of a piperazine derivative of formula (II) with a benzylhalogenide of formula (III) wherein X is defined in formula (I) and Y is a halomethylen group (—CH₂Br or CH₂Cl). The compound of formula (II) in an aprotic polar solvent, such as dimethylformamide, is reacted with an excess molar amount of a compound of formula (III) in the presence of a catalytic amount of potassium iodide. The resulting reaction mixture is stirred for about 3 hours to 24 hours at 60° C. in the presence of an acid-scavenging base, such as trimethylamine. The compound of formula (I) is then isolated from the reaction mixture by standard isolation techniques, such as organic phase extraction, evaporation of solvents and purification by flash column chromatography. Compounds of the general formula (II) can be prepared by following a protocol described e.g., in Sekiya et al., J. Med. Chem 1983, 26, 411. Compounds falling within the scope of formula (II) may be prepared by methods, which are generally analogous to those of said literature. Compounds of the general formula (III) are commercially available.

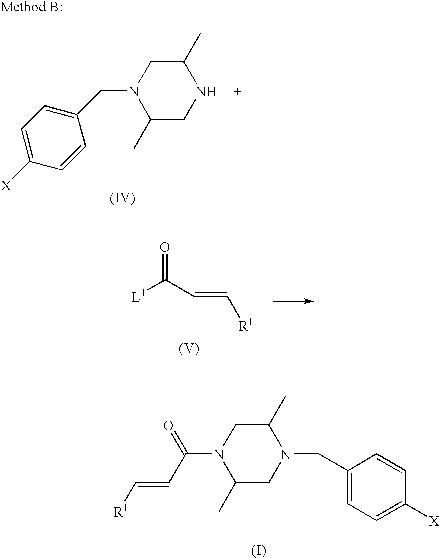

The compounds of formula (I) may also be prepared by treating the piperazine derivative of formula (IV), wherein X is defined in formula (I), with a compound of formula (V), wherein L¹ is a leaving group (e.g. a halide such as chloride, a hydroxyl, a benzotriazol-1-yl ester, an isourea group) and R¹ is defined in formula (I). The process of the invention may conveniently be carried out in an organic solvent such as CH₂Cl₂ or CHCl₃ at a temperature of, for example, 0° C. or above, such as 20 to 120° C.

Most preferred is a process where the amine derivative of formula (I) in chloroform is treated with an excess molar amount of a compound of formula (V), wherein L¹ is a hydroxy group, in the presence of an excess molar amount of a carbodiimide, such as N-cyclohexylcarbodiimide, N′-methylpolystyrene, and 1-hydroxybenzotriazol. The reaction mixture is stirred at a temperature typically in the range from 60° C. to 150° C. under a time typically in the range from 100 to 1000 seconds in a microwave oven (Smith Synthesiser from Personal Chemistry). Under these conditions the yields improve up to 99%. Compounds of formula (IV) may be obtained via a known protocol described e.g., in Tabia et al., J. Med. Chem. 1999, 42, 2870 or Example 9. Compounds falling within the scope of formula (I may be prepared by methods, which are generally analogous to those of said literature. Compounds of the formula (V) are commercially available or are described e.g., in Soloshonok et al., Helv. Chim. Acta 2002, 85, 3616; Anderson et al., J. Med. Chem. 1988, 31, 2097 and Larhed et al., J. Org. Chem. 1996, 61, 9582. Compounds falling within the scope of formula (V) may be prepared by methods, which are generally analogous to those of said literature or according to Example 1, Example 2, Example 3, and Example 4.

The present invention can also use acidic adducts of the dimethyl-piperazine derivatives where such acids include, for example, acids such as hydrochloric acid, hydrobromic acid, hydroiodic acid, sulfuric acid, phosphoric acid, carbonic acid, malic acid, citric acid, fumaric acid, tartaric acid, oxalic acid, methanesulfonic acid, p-toluenesulfonic acid, trifluoroacetic acid and others. Lists of additional suitable salts are found in Remington's Pharmaceutical Sciences, 17.th edition, Mack Publishing Company, Easton, Pa., 1985, p. 1418.

### EXAMPLE 1

### (E)-3-Chloro-4-nitro-cinnamic acid

A mixture of 2-chloro-4-bromo-aniline (413 mg, 2.0 mmol) and m-chloro-peroxybenzoic acid (60%, 1.72 g, 6 mmol) in dichloromethane (30 mL) was refluxed for 24 h. After cooling, the solution was washed with sodium carbonate (3×10 mL) and water (20 mL). The organic phase was evaporated and the residue was submitted to flash column chromatography to give 355 mg of 4-bromo-2-chloro-1-nitro-benzene (yield 75%). A solution of 4-bromo-2-chloro-1-nitrobenzene (237 mg, 1.0 mmol), acrylic acid methyl ester (108 μL, 1.2 mmol), potassium carbonate (150 mg, 1.1 mmol), tributylamine (263 μL, 1.1 mmol) and a catalytic amount of bis(triphenylphosphino)palladium(II) dichloride (0.005 eq.) in DMF (5 mL) was heated at 150° C. for 10 minutes in a microwave oven. 1M aqueous sodium hydroxide (1 mL) and water (4 mL) were added and the mixture was heated for 5 minutes at 130° C. in the microwave oven. The solution was acidified with 1N hydrochloric acid and the aqueous layer was extracted with ethyl acetate. The organic solvents were removed in vacuo and the residue was submitted to flash column chromatography to give the title compound in 40% yield.

¹H NMR: δ(CDCl₃) 7.94 (d, 1H), 7.72 (d, 1H), 7719 (d, 1H), 7.57 (dd, 2H), 6.56 (d, 1H)

Other cinnamic acids can be obtained in a similar mariner starting from aryl bromides or aryl iodides.

### EXAMPLE 2

### (E)-3-Benzo[2,1,3]oxadiazol-5-yl-acrylic acid

To a suspension of sodium hydride (29 mg, 1.2 mmol) in dry THF (6 mL) trietyl phosphonoacetate (269 mg, 1.2 mmol) was dropwise added and the reaction mixture was stirred at room temperature for 1 h. Benzo[2,1,3]oxadiazol-5-carbaldehyde (148 mg, 1.0 mmol) was added and the mixture was heated at 140° C. for 5 minutes in the microwave oven. 1 eq Wang-benzaldehyde resin was added and the mixture was heated for additionally 5 minutes at 140° C. in the microwave oven. Chloroform (6 mL) was added and the resin was filtered off and washed with chloroform. The organic solvent was evaporated and the residue submitted to flash column chromatography. The resulting ester was taken up in EtOH (3 mL) and 1M aqueous sodium hydroxide (1.5 mL) was added. The mixture was heated at 120° C. for 5 minutes in the microwave oven. The solution was acidified with 1N hydrochloric acid and the solid was filtered off, washed with water and dried to give the title compound in 73% yield.

¹H NMR: δ(CDCl₃) 8.36 (s, 1H), 8.05 (m, 2H), 7.74 (d, 1H), 6.82 (d, 1H)

Other (E)-acrylic acids can be obtained in a similar manner starting form aryl- or heteroaryl-aldehydes.

### EXAMPLE 3

### (E)-3-(5-Methoxy-benzo[2,1,3]oxadiazol-6-yl)-acrylic acid

1-Amino-5-methoxy-4-methyl-2-nitrobenzene in glacial AcOH (20 mL) was gradually added to ice-cooled stirred nitrosyl sulfuric acid [from NaNO₂ (11 mmol) and H₂SO₄ (20 mL sp gr 1.84)] such that the temperature did not exceed 15° C. When the addition was complete, stirring was continued for a further 1 h at 5° C., then the solution was poured onto crushed ice (100 g). Addition of this diazonium salt solution to NaN₃ (10 mmol) in H₂O (25 mL) participated the azide as a solid, which was not purified further because of the possibility of decomposition. The crude damp azide was refluxed in glacial AcOH (10 mL) for 1 h. After cooling, the solvent was evaporated to give 5-methoxy-6-methyl-benzofuroxan (yield 72%). To 5-methoxy-6-methyl-benzofuroxan (6.2 mmol) in refluxing EtOH (6 mL) was added dropwise P(OMe)₃ (12.4 mmol). When addition was complete (20 min) refluxing was continued for a further 1 h. The solvent was removed by rotary evaporation and the residue shaken with H₂O (10 mL). The solid obtained was filtered of and washed with water. The product was recrystallised from EtOH—H₂O to give 5-methoxy-6-methyl-benzo[2,1,3]oxadiazol (yield 64). 5-methoxy-6-methyl-benzo[2,1,3]oxadiazol (2.8 mmol), N-bromosuccinimide (3.1 mmol) and Bz₂O₂ (cat.) were refluxed in CCl₄ (6 mL) for 22 h. The cooled mixture was washed with H₂O (2×6 mL), the organic phase was dried (Na₂SO₄) and the solvent was evaporated. The residue was taken up in dioxan (8 mL) and calcium carbonate (14 mmol) and water (8 mL) were added. The mixture was refluxed for 3 h and then evaporated in vacuo. The residue was treated with CH₂Cl₂ and then with 2N hydrochloric acid until dissolution of the white precipitate occurred. The separated aqueous phase was extracted with CH₂Cl₂. The organic solvent were removed in vacuo and the residue was submitted to flash column chromatography (toluene→toluene:EtOAc, 20:1→toluene:EtOAc, 1:1) 6-hydroxymethyl-5-methoxy-benzo[2,1,3]oxadiazol (yield 61%). The alcohol (1.7 mmol) was dissolved in CHCl₃ (15 mL) and activated manganese dioxide (15 mmol) was added. The mixture was stirred at room temperature for 25 h and then filtered of through Celite. The filtrate was concentrated in vacuo to give 5-methoxy-benzo[2,1,3]oxadiazol-6-carbaldehyde (yield 86%). A mixture of 5-methoxy-benzo[2,1,3]oxadiazol-6-carbaldehyde (08 mmol), malonic acid (0.9 mmol), piperidine (5 μL), pyridine (0.5 mL) and EtOH (1.5 mL) was refluxed for 5 h under stirring. After cooling to room temperature the product precipitated. 2N hydrochloric acid was added and the mixture was stirred for 1 h. The precipitate was collected by filtration, washed with water, and dried under reduced pressure to give the title compound (yield 50%).

¹H NMR: δ(CDCl₃) 12.76 (bs, 1H), 8.49 (s, 1H), 7.77 (d, 1H), 7.32 (s, 1H), 6.81 (d, 1H), 4.00 (s, 3H).

(E)-3-(4-Bromo-benzo[2,1,3]oxadiazol-6-yl)-acrylic acid, (E)-3-benzo[2,1,3]oxadiazol-4-yl-acrylic acid and (E)-3-(5-chloro-benzo[2,1,3]ocadiazol-6-yl)-acrylic acid were prepared in a similar manner.

### EXAMPLE 4

### (E)-3-(4-Nitro-benzo[2,1,3]thiadiazol-5-yl)-acrylic acid

To a solution of 4-chloro-2-methyl-6-nitroaniline (5.0 g, 27 mmol) in 1,4-dioxane (20 mL) was added iron powder (5.2 g, 940 mmol) and aqueous NH₄Cl (5.0 g, 940 mmol in 13 mL of water). The reaction mixture was refluxed for five hours and then allowed to reach room temperature. The reaction mixture was filtered through Celite and was concentrated. The crude product was taken up into CH₂Cl₂, filtered and concentrated (yield of 3,4-diamino-2-nitrotoluene: 4.3 g, 99%).

To a solution oh 3,4-diamino-2-nitrotoluene (1.91 g, 11 mmol) in triethyl amine (7.7 mL) was added SOCl₂ (2.23 g, 19 mmol). The reaction mixture was refluxed for three hours and was then allowed to reach room temperature. The reaction mixture was filtered, concentrated and the residue was recrystallized from toluene/heptane (yield of 5-methyl-4-nitro-benzo[2,1,3]thiadiazole: 1.3 g, 61%).

¹H NMR: δ(CDCl₃) 8.11 (d, 1H), 769 (d, 1H), 2.67 (s, 3H).

To a solution of 5-methyl-4-nitro-benzo[2,1,3]thiadiazole (1.3 g, 6.7 mmol) in CCl₄ (10 mL) was added Br₂ (1.07 g, 6.7 mmol) and Bz₂O₂ (20 mg). The reaction mixture was refluxed for 120 hours, allowed to reach room temperature and was then evaporated to dryness. The residue was purified by flash chromatography using silica gel 60 and CH₂Cl₂/methanol (1:0→95:5) yielding a mixture of starting material and desired product (about 40%). This mixture was dissolved in 1,4-dioxane (10 mL). CaCO₃ (2.0 g, 20 mmol) and water (10 mL) were added and the reaction mixture was refluxed for 18 hours. The reaction mixture was allowed to reach room temperature and was concentrated to dryness. To a suspension of the remainder in CH₂Cl₂ (20 mL) 2M aqueous HCl was added until no solid remained The aqueous layer was extracted with CH₂Cl₂ and the combined organic layer was dried, filtered and concentrated. The crude product was dissolved in toluene and purified by flash chromatography using silica gel 60 and heptane/ethyl acetate (4:1→2:1→1:1) (yield of 5-(hydroxymethyl)-4-nitro-benzo[2,1,3]thiadiazole: 0.20 g, 14%).

¹H NMR: δ(CDCl₃) 7.94 (d, 1H), 7.83 (d, 1H), 4.97 (s, 2H), 2.04 (bs, 1H).

To a solution of 5-(hydroxymethyl)₄-nitro-benzo[2,1,3]thiadiazole (0.20 g, 0.95 mmol) in CHCl₃ (18 mL) was added MnO₂ (0.74 g, 8.5 mmol) and the reaction mixture was left at room temperature for 18 hours. The reaction mixture was filtered through Celite and was then concentrated (yield of 4-nitro-benzo[2,1,3]thiadiazol-5-yl-carbaldehyde: 0.18 g, 96%).

¹H NMR: δ(CDCl₃) 10.61 (s, 1H), 8.11 (d, 1H), 8.02 (d, 1H).

To a solution of 4-nitro-benzo[2,1,3]thiadiazol-5-yl-carbaldehyde (0.18 g, 086 mmol) in pyridine (2 mL) was added malonic acid (0.14 g, 1.34 mmol) and piperidine (0.1 mL). The reaction mixture was refluxed for 30 minutes and was then allowed to reach room temperature. The reaction mixture was acidified using 1M aqueous HCl and the precipitated crude product was collected by filtration and thoroughly washed with Cl₂Cl₂ (yield of (E)-3-(4-nitro-benzo[2,1,3]thiadiazol-5-yl)-acrylic acid: 0.043 g, 19%).

¹H NMR: δ(DMSO-d₆) 8.23 (d, 1H), 8.07 (m, 2H), 6.90 (d, 1H).

Other (E)-3-(benzo[2,1,3]thiadiazolyl)-acrylic acids were prepared in a similar manner.

### EXAMPLE 5

### 5.1 (E)-(trans)-3-(4-Bromo-phenyl)-1-[4-(4-fluoro-benzyl)-2,5-dimethyl-piperazine-1-yl]-prop-2-en-1-one

A mixture of (trans)-1-(4-fluoro-benzyl)-2,5-dimethyl-piperazine (222 mg, 1.0 mmol), (E)-3-(4-bromo-phenyl)-acrylic acid (341 mg, 1.5 mmol), 1-hydroxybenzotriazol (203 mg, 1.5 mmol) and N-cyclohexylcarbodiimide, N′-methylpolystyrene (167 g, 3.0 mmol of the resin with a loading of 1.8 mmol/g) in CHCl₃ was heated under 5 minutes at 110° C. in a microwave oven. The mixture was allowed to attain room temperature, TBD-methyl polystyrene (1003 mg, 3 mmol of the resin with a loading of 2.9 mmol/g) was added and the mixture was agitated over night. Both resins were filtered off and washed with CHCl₃ and EtOAc. The filtrate was concentrated in vacuo and the residue was submitted to flash column chromatography (toluene toluene:EtOAc, 20:1→toluene:EtOAc, 1:1) to give the title product in 97% yield.

¹H NMR: δ(CDCl₃) 7.61 (d, 1H), 7.50 (dd 2H), 7.37 (d, 2H), 7.33 (dd, 2H, 7.01 (dd, 2H), 6.84 (d, 1H), 3.53 (m, 2H), 3.06 (bs, 1H), 2.74 bs, 1H), 2.29 (d, 1H), 1.34 (d, 3H), 1.01 (d, 3H)

The following compounds were prepared in a similar manner:

### 5.2 (E)-(trans)-3-(4-Chloro-3-nitro-phenyl)-1-[4-(4-chloro-benzyl)-2,5-dimethyl-piperazine-1-yl]-prop-2-en-1-one

¹H NMR: δ(CDCl₃) 8.01 (dd, 1H), 7.59 (m, 3H), 7.30 (m, 41), 6.92 (d, 1H), 3.54 (m, 2H), 3.08 (bs, 1H), 2.76 (d, 1H), 2.30 (d, 1H), 31.35 (d, 3H), 1.01 (d, 3H)

### 5.3 (E)-(trans)-3-(3,4-Dichloro-phenyl)-1-[4-(4-fluoro-benzyl)-2,5-dimethyl-piperazine-1-yl]-prop-2-en-1-one

¹H NMR: δ(CDCl₃) 7.59 (m, 1H), 7.43 (m, 1H), 7.33 (m, 3H), 7.01 (dd, 2H) 6.83 (d, 1H), 3.53 (m, 2H), 3.07 (bs, 1H), 2.74 (d, 1H), 2.29 (d, 1H), 1.34 (bs, 3H), 1.01 (d, 3H)

### 5.4 (E)-(trans)-1-[4-(4-Fluoro-benzyl)-2,5-dimethyl-piperazine-1-yl-3-p-tolyl-prop-2-en-1-one

¹H NMR: δ(CDCl₃) 7.66 (d, 1H), 7.41 (d, 2H), 7.32 (m, 2H), 7.17 (d, 2H), 7.01 (dd, 2H, 6.81 (d, 1H), 3.52 (m, 2H), 3.05 (bs, 1H), 2.74 (dd, 1H), 2.36 (s, 31), 2.28 (dd, 1H), 1.33 (d, 3H), 1.01 (d, 3H)

### 5.5 (E)-(trans)-1-[4-(4-Fluoro-benzyl)-2,5-dim ethyl-piperazine-1-yl]-3-(4-nitro-phenyl)-prop-2-en-1-one

¹H NMR: δ(CDCl₃) 8.23 (d, 2H), 7.70 (d, 1H), 7.65 (d, 2H), 7.33 (dd, 2H), 7.02 (dd, 2H), 6.97 (d, 1H), 3.54 (m, 3H), 3.08 (bs, 1H), 2.76 (d, 1H), 2.31 (d, 1H), 1.36 (bs, 39, 1.02 (d,

### 5.6 (E)-(trans)-3-(2,4-Dichloro-phenyl)-]-[4-(4-fluoro-benzyl)-2,5-dimethyl-piperazine-1-yl]-prop-2-en-1-one hydrochloride

¹H NMR: δ(CDCl₃) 7.98 (d, 1H), 7.91 (dd, 1H), 7.49 (m, 2H), 7.29 (m, 2H), 7.15 (dd, 2H), 6.74 (d, 1H), 4.38 (m, 2H), 3.93 (m, 2H), 2.97 (bs, 1H), 2.85 (d, 1H), 1.63 (bs), 1.40 (d, 3H)

### 5.7 (E)-(trans)-3-Benzo[b]thiophen-2-yl-1-[4-(4-fluoro-benzyl)-2,5-dimethyl-piperazine-1-yl]-prop-2-en-1-one

¹H NMR: δ(CDCl₃) 7.90 (d, 1H), 7.76 (m, 2H), 7.42 (s, 1H), 7.34 (m, 4H), 7.01 (d, 2H), 6.71 (d, 1H), 3.53 (m, 2H), 3.07 (bs, 1H), 2.75 (d, 1H), 2.30 (d, 1H), 1.34 (bs, 3H), 1.02 (d, 3H)

### 5.8 (E)-(trans)-3-Benzo[b]thiophen-3-yl-1-[4-(4-fluoro-benzyl)-2,5-dimethyl-piperazine-1-yl]-prop-2-en-1-one

¹H NMR: δ(CDCl₃) 8.01 (s, 1H), 7.98 (d, 2H), 7.88 (d, 1H), 7.69 (s, 1H), 7.43 (m, 2H), 7.33 (dd, 2H), 7.01 (dd, 2H), 6.95 (d, 1H), 3.54 (m, 2H), 3.07 (bs, 1H), 2.77 (dd, 1H), 2.31 (d, 1H), 1.36 (d, 3H), 1.03 (d, 3H)

### 5.9 (E)-(trans)-3-(3,4-Dichloro-phenyl)-1-(4-(4-chloro-benzyl)-2,5-dimethyl-piperazine-1-yl]-prop-2-en-1-one

¹H NMR: δ(CDCl₃) 7.57 (m, 2H), 7.44 (d, 1H), 7.31 (m, 5H), 6.83 (d, 1H), 3.53 (m, 2H), 3.07 (bs, 1H), 2.75 (d, 1H), 2.29 (d, 1H), 1.34 (bs, 3H), 1.01 (d, 3H)

### 5.10 (E)-(trans)-3-(3,4-Dimethoxy-phenyl)-1-[4-(4-fluoro-benzyl)-2,5-dimethyl-piperazine-1-yl]-prop-2-en-1-one

¹H NMR: δ(CDCl₃) 7.63 (d, 1H), 7.33 (dd, 2H), 7.11 (dd, 1H), 7.01 (m, 3H), 6.86 (d, 1H), 6.71 (d, 1H), 3.92 (s, 3H), 3.91 (s, 3H), 3.53 (m, 2H), 3.05 (bs, 1H), 2.75 (dd, 1H), 2.29 (dd, 1H), 1.34 (d, 3H), 1.01 (d, 31)

### 5.11 (E)-(trans)-3-(3-Bromo-4,5-dimethoxy-phenyl)-]-[4-(4-fluoro-benzyl)-2,5-dimethyl-piperazine-1-yl]-prop-2-en-1-one

¹HNMR: δ(CDCl₃) 7.54 (d, 1H), 7.33 (n, 3H), 7.01 (dd, 2H), 6.93 (d, 1H), 6.75 (d, 1H), 3.89 (s, 3H), 3.88 (s, 3H), 3.53 (m, 2H), 3.06 (bs, 1H), 2.75 (d, 1H), 2.29 (d, 1H), 1.34 (d, 3H), 1.01 (d, 3H)

### 5.12 (E)-(trans)-3-(4-Chloro-3-trifluoromethyl-phenyl)-1-[4-(4-fluoro-benzyl)-2,5-dimethyl-piperazine-1-yl]-prop-2-en-1-one

¹H NMR: δ(CDCl₃), 7.80 (d, 1H), 7.63 (d, 1H), 7.51 (d, 1H), 7.33 (dd, 2H), 7.01 (dd, 2H), 6.88 (d, 1H), 3.53 (m, 2H), 3.07 (bs, 1H), 2.75 (d, 1H), 2.30 (d, 1H), 1.35 (bs, 3H), 1.01 (d, 3H)

### 5.13 (E)-(trans)-3-Benzo[2,1,3]oxadiazol-5-yl-1-[4-(4-fluoro-benzyl)-2,5-dimethyl-piperazine-1-yl]-prop-2-en-1-one

¹H NMR: δ(CDCl₃) 7.89 (s, 1H), 7.85 (d, 1H), 7.72 (d, 1H), 7.62 (d, 1H), 7.38 (dd, 2H), 7.03 (m, 3H), 3.63 (m, 2H), 3.21 (bs, 1H), 2.81 (bs, 1H), 2.38 (d, 1H), 1.38 (d, 3H), 1.07 (d, 3H)

### 5.14 (E)-(trans)-3-(2,4-Dim ethyl-phenyl)-1-[4-(4-fluoro-benzyl)-2,5-dimethyl-piperazine-1-yl]-prop-2-en-1-one

¹H NMR: δ(CDCl₃) 7.94 (d, 1H), 7.44 (d, 1H), 7.34 (dd, 2H), 7.02 (m, 4H), 6.73 (d, 1H), 3.54 (m, 2H), 3.06 (bs, 1H), 2.75 (dd, 1H), 2.41 (s, 3H), 2.33 (s, 3H), 2.29 (d, 1H), 1.34 (d, 3H), 1.02 (d, 3H)

### 5.15 (E)-(trans)-1-[4-(4-Chloro-benzyl)-2,5-dim ethyl-piperazine-1-yl]-3-(4-chloro-phenyl)-prop-2-en-1-one

¹H NMR: δ(CDCl₃) 7.63 (d, 1H), 7.44 (d, 2H), 7.34 (m, 61), 6.82 (d, 1H), 3.53 (m, 2H), 3.06 (bs, 1H), 2.75 (dd, 1H), 2.28 (d, 1H), 1.34 (d, 3H), 1.01 (d, 3H)

### 5.16 (E)-(trans)-1-[4-(4-Fluoro-benzyl)-2,5-dimethyl-piperazine-1-yl]-3-(4-methyl-3-nitro-phenyl)-pr-op-2-en-1-one

¹H NMR: δ(CDCl₃) 8.12 (d, 1H), 7.63 (m, 2H), 7.34 (m, 3H), 7.03 (dd, 2H), 6.91 (d, 1H), 3.54 (m, 2H), 3.07 (bs, 1H), 2.75 (d, 1H), 2.62 (s, 3H), 2.30 (d, 1H), 1.35 (bs, 3H), 1.01 (d,

### 5.17 (E)-(trans)-1-[4-(4-Chloro-enzyl)-Z 5-dimethyl-piperazine-1-yl]-3-(4-methyl-3-nitro-phenyl)-prop-2-en-1-one

¹H NMR: δ(CDCl₃) 8.13 (d, 1H), 7.65 (d, 1H), 7.59 (dd, 1H), 7.35 (d, 1H), 7.30 (m, 4H), 6.91 (d, EH), 3.54 (m, 2H), 3.08 (bs, 1H), 2.76 (bs, 1H), 2.62 (s, 3H), 2.29 (d, 1H), 1.35 (bs, 3H), 1.01 (d, 3H)

### 5.18 (E)-(trans)-3-Benzo[2,1,3]thiadiazol-5-yl-]-[4-(4-fluoro-benzyl)-2,5-dimethyl-piperazine-1-yl]-prop-2-en-1-one

¹H NMR: δ(CDCl₃) 8.08 (s, 1H), 7.99 (d, 1H), 7.81 (dd, 2H), 7.34 (dd, 2H), 7.02 (m, 3H), 3.55 (m, 2H), 3.09 (bs, 1H), 2.77 (d, 1H), 2.32 (d, 1H), 1.37 (bs, 3H), 1.04 (d, 3H)

### 5.19 (E)-(trans)-1-[4-(4-Fluoro-benzyl)-2,5-dimethyl-piperazine-1-yl]-3-(3,4,5-trimethoxy-phenyl)-prop-2-en-1-one

¹H NMR: δ(CDCl₃) 7.59 (d, 1H), 7.33 (m, 2H), 7.01 (dd, 2H), 6.73 (m, 3H), 3.89 (s, 6H), 3.87 (s, 3H), 3.53 (T, 2H), 3.06 (bs, 1H), 2.75 (dd, 1H), 2.29 (dd, 1H), 1.34 (d, 3H), 1.01 (d, 3H)

### 5.20 (F)-(trans)-3-(4-Chloro-2-nitro-phenyl)-1-[4-(4-fluoro-benzyl)-2,5-dimethyl-piperazine-1-yl]-prop-2-en-1-one

¹H NMR: δ(CDCl₃) 8.03 (d, 1H), 7.85 (d, 1H), 7.58 (m, 2H), 7.33 (dd, 2H), 7.01 (dd, 2H), 6.69 (d, 1H), 3.54 (m, 2H), 3.07 (bs, 1H), 2.76 (dd, 1H), 2.30 (dd, 1H), 1.35 (d, 3H), 1.03 (d, 3H)

### 5.21 (E)-(trans)-3-(3-Chloro-4-nitro-phenyl)-1-[4-(4-fluoro-benzyl)-2,5-dimethyl-piperazine-1-yl]-prop-2-en-1-one

¹HNMR: δ(CDCl₃) 7.89 (d, 1H) 7.64 (m, 1H), 7.59 (d, 1H), 7.48 (dd, 1H), 7.31 (dd, 2H), 699 (m, 3H), 3.52 (m, 2H), 3.06 (bs, 1H), 2.72 (bs, 1H), 2.29 (d, 1H), 1.34 (bs, 3H), 0.99 (d, 3H); MS: (ESI) 432 [M+H]⁺.

### 5.22 (E)-(trans)-3-(4-Chloro-3-methoxy-5-nitro-phenyl)-1-[4-(4-fluoro-benzyl)-2,5-dimethyl-piperazine-1-yl]-prop-2-en-1-one

¹H NMR: δ(CDCl₃) 7.58 (d, 1H), 7.54 (d, 1H), 7.31 (dd, 2H), 7.12 (d, 1H), 7.00 (dd, 2H), 6.88 (d, 1H), 3.98 (s, 3H), 3.52 (m, 2H), 3.06 (bs, 1H), 2.74 (d, 1H), 2.29 (d, 1H), 1.33 (bs, 3H), 0.99 (d, 3H); MS: (ESI) 462 [M+H]⁺.

### 5.23 (E)-(trans)-1-[4-(4-Fluoro-benzyl)-2,5-dimethyl-piperazine-1-yl]-3-(4-trifluoromethyl-phenyl)-prop-2-en-1-one

¹H NMR: δ(CDCl₃) 7.62 (d, 1H), 7.55 (m, 4H), 7.26 (dd, 2H), 6.96 (m, 2H), 6.86 (d, 1H), 3.47 (m, 2H), 3.01 (bs, 1H), 2.69 (d, 1H), 2.23 (d, 1H), 1.28 (bs, 3H), 0.95 (d, 3H).

### 5.24 (E)-(trans)-3-(2-Chloro-4-methyl-phenyl)-1-[4-(4-fluoro-benzyl)-2,5-dimethyl-piperazine-1-yl]-prop-2-en-1-one

¹H NMR: δ(CDCl₃) 7.98 (d, 1H), 748 (d, 1H), 7.34 (dd, 2H), 7.24 (s, 1H), 7.07 (d, 1H), 7.02 (dd, 2H), 6.81 (d, 1H), 3.54 (m, 2H), 3.06 (bs, 1H), 2.75 (dd, 1H), 2.35 (s, 3H), 2.29 (dd, 1H), 1.35 (d, 3H), 1.02 (d, 3H).

### 5.25 (E)-(trans)-1-[4-(4-Fluoro-benzyl)-2,5-dimethyl-piperazine-1-yl]-3-(3-trifluoromethyl-4-nitro-phenyl)-prop-2-en-1-one

7.93 (m, 2H), 7.81 (d, 1H), 7.69 (d, 1H), 7.34 (dd, 2H), 7.02 (m, 3H), 3.55 (m, 2H), 3.10 (bs, 1H), 2.76 (bs, 1H), 2.32 (d, 1H), 1.37 (bs, 3H), 1.03 (d, 3H).

### 5.26 (E)-(trans)-3-(4-Chloro-3-methoxy-phenyl)-1-[4-(4-fluoro-benzyl)-2,5-dimethyl-piperazine-1-yl]-prop-2-en-1-one

¹H NMR: δ(CDCl₃) 7.63 (d, 1H), 7.35 (m, 3H), 7.10 (dd, 1H), 7.03 (dd, 2H), 6.83 (d, 1H), 3.94 (s, 3H), 3.54 (m, 2H), 3.08 (bs, 1H), 2.76 (dd, 1H), 2.32 (d, 1H), 1.36 (d, 3H), 1.02 (d, 3H).

### 5.27 (E)-(trans)-3-(3-Chloro-4,5-dimethoxy-phenyl)-1-[4-(4-fluorobenzyl)-2,5-dimethyl-piperazine-1-yl]-prop-2-en-1-one

¹H NMR: δ(CDCl₃) 7.56 (d, 1H), 7.34 (dd, 2H), 7.19 (d, 1H), 7.02 (dd, 2H), 6.91 (d, 1H), 6.77 (d, 1H), 3.91 (s, 3H), 3.90 (s, 3H), 3.54 (m, 2H), 3.07 (bs, 1H), 2.76 (m, 1H), 2.30 (d, 1H), 1.35 (d, 3H), 1.02 (d, 3H).

### 5.28 (E)-(trans)-1-[4-(4-Fluorobenzyl)-2,5-dim ethyl-piperazine-1-yl]-3-(5-methoxy-benzo[2,1,3]oxadiazol-6-yl)-prop-2-en-1-one

¹H NMR: δ(CDCl₃) 7.88 (m, 2H), 7.33 (dd, 2H), 7.01 (m, 3H), 6.90 (s, 1H), 3.97 (s, 3H), 3.54 (m, 2H), 3.08 (bs, 1H), 2.76 (d, 1H), 2.31 (d, 1H), 1.36 (bs, 3H), 1.03 (d, 3H).

### 5.29 (E)-(trans)-3-(4-Bromo-3,5-dimethoxy-phenyl)-1-[4-(4-fluorobenzyl)-2,5-dimethyl-piperazine-1-yl]-prop-2-en-1-one

¹H NMR: δ(CDCl₃) 7.61 (d, 1H), 7.34 (dd, 2H), 7.02 (dd, 2H), 6.85 (d, 1H), 6.70 (s, 2H), 394 (s, 6H), 354 (m, 2H), 3.08 (bs, 1H), 2.77 (dd, 1H), 2.31 (d, 1H), 1.36 (d, 3H), 1.02 (d, 3H).

### 5.30 (E)-(trans)-1-[4-(4-Fluorobenzyl)-2,5-dimethyl-piperazine-1-yl]-3-(3-methoxy-4-methyl-phenyl)-prop-2-en-1-one

¹H NMR: δ(CDCl₃) 7.66 (d, 1H), 7.34 (dd, 2H), 7.14 (d, 1H), 7.07 (d, 1H), 7.02 (dd, 2H), 694 (s, 1H), 681 (d, 1H), 3.87 (s, 3H), 3.54 (m, 2H), 3.07 (bs, 1H), 2.76 (dd, 1H), 2.30 (d, 1H), 2.24 (s, 31), 1.35 (d, 3H), 1.02 (d, 3H).

### 5.31 (E)-(trans)-3-Benzo[2,1,3]oxadiazol-4-yl-1-[4-(4-fluorobenzyl)-2,5-dimethyl-piperazine-1-yl]-prop-2-en-1-one

¹H NMR: δ(CDCl₃) 7.93 (bs, 1H), 7.79 (m, 2H), 7.45 (m, 2H), 7.35 (dd, 2H), 7.02 (dd, 2H), 4-37 (bs, 1H), 3.79 (bs, 1H), 3.55 (m, 2H), 3.10 (d, 2H), 2.78 (s, 1H), 2.34 (bs, 1H), 1.39 (d, 3H), 1.04 (d, 3H).

### 5.32 (E)-(trans)-3-(4-Bromo-benzo[2,1,3]oxadiazol-6-yl)-1-[4-(4-fluorobenzyl)-2,5-dimethyl-piperazine-1-yl]-prop-2-en-1-one

¹H NMR: δ(CDCl₃) 7.83 (s, 2H), 7.68 (d, 1H), 7.34 (dd, 2H), 7.01 (m, 3H), 3.55 (m, 2H), 3.11 (bs, 1H), 2.78 (d, 1H), 2.33 (d, 1H), 1.38 (bs, 3H), 1.04 (d, 3H).

### 5.33 (E)-(trans)-3-(4-Bromo-phenyl)-1-[4-(4-chlorobenzyl)-2,5-dimethyl-piperazine-1-yl]-prop-2-en-1-one

¹H NMR: δ(CDCl₃) 7.61 (d, 1H), 7.50 (d, 2H), 7.37 (d, 2H), 7.30 (m, 4H), 6.84 (d, 1H), 3.53 (m, 2H), 3.06 (bs, 1H), 2.74 (d, 1H), 2.28 (d, 1H), 1.34 (d, 3H), 1.00 (d, 3H).

### 5.34 (E)-(trans)-3-(3-Bromo-4,5-dimethoxy-phenyl)-1-[4-(4-chlorobenzyl)-2,5-dimethyl-piperazine-1-yl]-prop-2-en-1-one

¹H NMR: δ(CDCl₃) 7.54 (d, 1H), 7.34 (d, 1H), 730 (m, 4H), 6.93 (d, 1H), 6.74 (d, 1H), 3.89 (s, 3H), 3.88 (s, 3H), 3.53 (m, 2H), 3.06 (bs, 1H), 2.75 (d, 1H), 2.28 (d, 1H), 1.34 (d, 3H), 1.01 (d, 3H).

### 5.35 (E)-(trans)-1-[4-(4-Chlorobenzyl)-2,5-dimethyl-piperazine-1-yl]-3-(4-chloro-3-trifluoromethyl-phenyl)-prop-2-en-1-one

¹H NMR: δ(CDCl₃) 7.80 (d, 1H), 7.63 (d, 1H), 7.58 (dd, 1H), 7.51 (d, 1H), 7.30 (m, 4H), 6.88 (d, 1H), 3.53 (m, 2H), 3.07 (bs, 1H), 2.75 (d, 1H), 2.29 (d, 1H), 1.35 (bs, 3H), 1.01 (d, 3H).

### 5.36 (E)-(trans)-1-[4-(4-Chlorobenzyl)-2,5-dimethyl-piperazine-1-yl]-3-(4-nitro-phenyl)-prop-2-en-1-one

¹H NMR: δ(CDCl₃) 8.23 (d, 2H), 7.70 (d, 1H), 7.65 (d, 2H), 7.30 (m, 4H), 6.98 (d, 1H), 3.54 (m, 2H), 3.08 (bs, 1H), 2.76 (d, 1H), 2.30 (d, 1H), 1.36 (bs, 3H), 1.02 (d, 3H).

### 5.37 (E)-(trans)-1-[4-(4-Chlorobenzyl)-2,5-dimethyl-piperazin e-1-yl]-3-(4-methyl-phenyl)-prop-2-en-1-one

¹HNMR: δ(CDCl₃) 7.66 (d, 1H), 7.41 (d, 2H), 7.30 (m, 41-1H), 717 (d, 2H), 6.81 (d, 1H), 3.53 (m, 2H), 3.05 (bs, 1H), 2.74 (dd, 1H), 2.37 (s, 31-1H), 2.27 (dd, 1H), 1.33 (d, 3H), 1.00 (d, 3H).

### 5.38 (E)-(trans)-3-(5-chloro-benzo[2,1,3]oxadiazol-6-yl)-1-[4-(4-fluorobenzyl)-2,5-dimethyl-piperazine-1-yl]-prop-2-en-1-one

¹H NMR: δ(CDCl₃) 8.03 (s, 1H), 7.96 (m, 2H), 7.33 (dd, 2H), 7.02 (m, 2H), 6.94 (bs, 1H), 3.54 (m, 2H), 3.09 (s, 1H), 2.76 (bs, 1H), 2.31 (d, 1H), 1.37 (bs, 3H), 1.04 (d, 3H).

### 5.39 (E)-(trans)-3-(3-Bromo-4-chloro-phenyl)-1-[4-(4-fluorobenzyl)-2,5-dimethyl-piperazine-1-yl]-prop-2-en-1-one

¹H NMR: δ(CDCl₃) 7.77 (d, 1H), 758 (d, 1H), 7.45 (d, 1H), 7.37 (d, 1H), 7.32 (dd, 2H), 7.02 (t, 21-1H), 6.83 (bd, 1H), 3.61 (d, 1H), 3.46 (d, 1H), 3.07 (bs, 1H), 2.75 (bd, 1H), 2.30 (d, 1H), 1.34 (bs, 3H), 1.01 (d, 3H).

### 5.40 (E)-(trans)-3-(4-Bromo-3-chloro-phenyl)-1-[4-(4-fluorobenzyl)-2,5-dimethyl-piperazine-1-yl]-prop-2-en-1-one

¹H NMR: δ(CDCl₃) 7.58 (m, 3H), 7.33 (dd, 2H), 7.24 (d, 1H), 7.02 (t, 2H), 6.87 (bd, 1H), 3.61 (d, 1H), 3.46 (d, 1H), 3.07 (bs, 1H), 2.75 (bd, 1H), 2.29 (d, 1H), 1.26 (bs, 3H), 1.01 (d, 3H).

### 5.41 (E)-(trans)-3-(3,4-Dibromo-phenyl)-1-[4-(4-fluorobenzyl)-2,5-dimethyl-piperazine-1-yl]-prop-2-en-1-one

¹H NMR: δ(CDCl₃) 7.76 (d, 1H), 7.61 (d, 1H), 7.54 (d, 1H), 7.33 (dd, 2H), 7.27 (m, 1H), 7.02 (t, 2H), 6.86 (bd, 1H), 3.61 (d, 1H), 3.46 (d, 1H), 3.07 (bs, 1H), 2.75 (bd, 1H), 2.30 (d, 1H), 1.34 (bs, 3H), 1.01 (d, 3H).

### 5.42 (E)-(trans)-3-(4-Bromo-3-nitro-phenyl)-1-[4-(4-fluorobenzyl)-2,5-dim ethyl-piperazine-1-yl-prop-2-en-1-one

¹H NMR: δ(CDCl₃) 7.97 (d, 1H), 7.74 (d, 1H), 7.63 (d, 1H), 7.52 (d, 1H), 7.33 (dd, 2H), 7.02 (t, 2H), 6.94 (bd, 1H), 3.61 (d, 1H), 3.46 (d, 1H), 3.08 (bs, 1H), 2.75 (bd, 1H), 2.31 (d, 1H), 1.32 (bs, 3H), 1.01 (d, 3H).

### 5.43 (E)-(trans)-3-(4-Chloro-benzo[2,1,3]oxadiazol-6-yl)-1-[4-(4-fluorobenzyl)-2,5-dimethyl-piperazine-1-yl]-prop-2-en-1-one

¹H NMR: δ(CDCl₃) 7.80 (s, 1H), 7.68 (d, 1H), 7.63 (s, 1H), 7.33 (dd, 2H), 6.99 (m, 3H), 3.62 (d, 1H), 3.48 (d, 1H), 3.09 (bs, 1H), 2.77 (bs, 1H), 2.32 (d, 1H), 1.33 (bs, 3H), 1.03 (d, 3H).

### 5.44 (E)-(trans)-3-(6-Chloro-benzo[2,1,3]oxadiazol-4-yl)-1-[4-(4-fluorobenzyl)-2,5-dimethyl-piperazine-1-yl]-prop-2-en-1-one

¹H NMR: δ(CDCl₃) 7.96 (bt, 1H), 7.82 (d, 1H), 7.69 (d, 1H), 7.39 (d, 1H), 7.34 (dd, 2H), 7.02 (dd, 2H), [4.88 (bs) and 3.33 (bs) (1H)], 4.34 (bd, 1H), 3.76 (bs, 1H), 3.62 (d, 1H), 3.48 (d, 1H), 310 (bs, 1H), 2.80 (bs, 1H), 2.33 (bs, 1H), 1.35 (bs, 3H), 1.03 (d, 3H)

### 5.45 (E)-(trans)-3-(4-Bromo-benzo[2,1,3]thiadiazol-6-yl)-1-[4-(4-fluorobenzyl)-2,5-dimethyl-piperazine-1-yl]-prop-2-en-1-one

¹H NMR: δ(CDCl₃) 8.04 (s, 1H, 8.01 (s, 1H), 7.77 (d, 1H), 7.34 (dd, 2H), 7.01 (m, 3H), 3.62 (d, 1H), 3.48 (d, 1H), 3.10 (bs, 1H), 2.78 (bs, 1H), 2.31 (d, 1H), 1.31 (bs, 3H), 1.04 (d, 3H).

### 5.46 (E)-(trans)-3-(4-Chloro-benzo[2,1,3]thiadiazol-6-yl)-1-[4-(4-fluorobenzyl)-2,5-dimethyl-piperazine-1-yl]-prop-2-en-1-one

¹H NMR: δ(CDCl₃) 7.98 (s, 1H), 784 (s, 1H), 7.78 (d, 1H), 7.34 (dd, 2H), 7.02 (m, 3H), 3.62 (d, 1H), 3.47 (d, 1H), 3.10 (bs, 1H), 2.78 (bd, 1H), 2.32 (d, 1H), 1.31 (bs, 3H), 1.04 (d, 3H).

### 5.47 (E)-(trans)-3-(4-Bromo-5-methoxy-benzo[2,1,3]thiadiazol-6-yl)-1-[4-(4-fluorobenzyl) 2,5-dimethyl-piperazine-1-yl]-prop-2-en-1-one

¹H NMR: δ(CDCl₃) 8.10 (s, 1H), 7.94 (d, 1H), 7.34 (dd, 2H), 7.02 (m, 3H), 3.98 (s, 3H), 3.62 (d, 1H), 3.47 (d, 1H), 3.09 (bs, 1H), 2.78 (bs, 1H), 2.32 (d, 1H), 131 (bs, 3H), 1.04 (d, 3H).

### 5.48 (E)-(trans)-1-[4-(4-Fluoro-benzyl)-2,5-dimethyl-piperazine-1-yl]-3-(4-nitro benzo[2,1,3]thiadiazol-5-yl)-prop-2-en-1-one

¹H NMR: δ(CDCl₃) 8.17 (d, 1H), 7.93 (d, 1H), 7.81 (d, 1H), 7.34 (dd, 2H), 7.02 (m, 3H), 3.63 (d, 1H), 3.48 (d, 1H), 3.09 (bs, 1H), 2.78 (bd, 1H), 2.32 (d, 1H), 1.38 (bs, 3H), 1.05 (d, 3H).

### 5.49 (E)-(trans)-3-(6-Chloro-benzo[2,1,3]thiadiazol-4-yl)-]-[4-(4-fluorobenzyl)-2,5-dimethyl-piperazine-1-yl]-prop-2-en-1-one

¹HNMR: δ(CDCl₃) 8.17 (bd, 1H), 7.98 (d, 1H), 7.83 (bd, 1H), 7.61 (d, 1H), 7.34 (dd, 2H), 7.02 (t, 2H), [4.89 (bs) and 3.33 (bs) (1H)], 4.37 (bs, 1H), 3.78 (bs, 1H), 3.62 (d, 1H), 3.48 (d, 1H), 3.10 (bs, 1H), 2.79 (bs, 1H), 2.34 (bd, 1H), 1.38 (bs, 3H), 1.04 (d, 3H).

### EXAMPLE 6

### 6.1 (E)-(trans)-3-(4-Chloro-phenyl)-1-[4-(4-fluoro-benzyl)-2,5-dimethyl-piperazine-1-yl]-prop-2-en-1-one

To a solution of (trans)-1-(4-fluoro-benzyl)-2,5-dimethyl-piperazine (1.30 g, 6.7 mmol) and trimethylamine (1.41 mL, 10.1 mmol) in 15 mL of CHCl₃, a solution of (E)-4-chloro-cinnamoyl-chloride (1.34 g, 6.7 mmol) was added and the reaction mixture was stirred for 3 h at room temperature. The organic layer was washed with 1M aqueous NaOH, dried and concentrated. The residue was recrystallised from EtOH:water (7:3) to give the pure product in 80% yield.

¹H NMR: δ(d6-acetone) 7.66 (m, 2H), 7.54 (d, 1H), 7.40 (m, 4H), 7.22 (d, 1H), 7.06 (m, 2H), 4.59 (bs, 1H); 4.10 (bs, 1H), 3.56 (m, 2H), 3.05 (bs, 1H), 2.73 (d, 1H), 2.29 (dd, 1H), 1.27 (d, 3H), 0.97 (d, 3H)

The following compound was prepared in a similar manner:

### 6.2 (E)-(trans)-3-(4-Chloro-3-nitro-phenyl)-1-[4-(4-fluoro-benzyl)-2,5-dimethyl-piperazne-1-yl]-prop-2-en-1-one

¹H NMR: δ(d6-acetone) 8.29 (d, 1H), 7.95 (dd, 1H), 7.71 (d, 1H), 7.59 (d, 1H), 7.42 (m, 3H), 7.06 (m, 2 μl), 4.60 (bs, 1H), 4.11 (bs, 1H), 3.56 (m, 2H), 3.05 (bs, 1H), 2.73 (bs, 1H), 2.29 (d, 1H), 127 (bs, 3H), 0.97 (d, 3H).

### EXAMPLE 7

### p-Chloro-cis-cinnamic acid

A solution of 18-crown-6 (5.0 g, 18.9 mmol) in THF (20 mL) was cooled to 40° C. and bis(2,2,2-trifluoroethyl)-(methoxycarbonylmethyl)phosphonate (0.85 mL, 4 mmol) followed by KHMDS (890 mg, 4 mmol) were added. After stirring for 15 min, p-chlorobenzaldehyde (560 mg, 3.78 mmol) was added and the solution was stirred for 2 h. Saturated aqueous ammonium chloride (50 mL) and ethyl ether (30 mL) were added and the organic phase was washed with 1 N HCl. After drying and evaporation, the residue was purified by chromatography (SiO₂, H/E 4/1) to give p-chloro-cis-cinnamic acid methyl ester (610 mg, 82%). The methyl ester was hydrolysed in EtOH/aqueous 1 M NaOH; 2/1 (15 mL) at 120° C. for 5 min. After addition of water and aqueous HCl the precipitate was collected to afford pure p-chloro-cis-cinnamic acid (402 mg, 71%).

¹H NMR: δ(CDCl₃) 11.26 (bs, 1H), 7.54 (m, 2H), 7.32 (m, 2H), 7.00 (d, 1H), 5.97 (d, 1H).

### EXAMPLE 8

In the same manner as described in Example 5, the p-chloro-cis-cinnamic acid was reacted with

### a) (trans)-1-(4-fluoro-benzyl)-2,5-dimethyl-piperazine

### 8.1 (Z)-(trans)-3-(4-Chloro-phenyl)-1-[4-(4-fluoro-benzyl)-2,5-dimethyl-piperazine-1-yl]-prop-2-en-1-one; compound D

¹H NMR: δ(CDCl₃) 7.43 (d, 1H, rotamer A), 737 (d, 1H, rotamer B), 7.28 (m, 1H), rotamer A+B), 6.96 (m, 4H, rotamer A+B), 6.56 (d, 1H, rotamer A), 6.55 (d, 1H, rotamer B), 6.06 (d, 1H, rotamer A), 6.02 (d, 1H, rotamer B), 4.77 (m, 1H, rotamer B), 427 (d, 1H, rotamer), 3.97 (m, 1H, rotamer A), 3.53 (m, 2H, rotamer A+B), 3.37 (m, 3H, rotamer A+B), 3.28 (dd, 1H, rotamer B), 3.16 (dd, 1H, rotamer A), 3.04 (m, 1H, rotamer A), 2.83 (m, 1H, rotamer 13), 2.64 (dd, 1H, rotamer B), 2.39 (dd, 1H, rotamer A), 2.21 (d, 1H, rotamer B), 2.07 (d, 1H, rotamer A), 1.22 (d, 3H, rotamer B), 1.12 (d, 3H, rotamer A), 0.93 (d, 3H, rotamer A), 0.80 (d, 3H, rotamer B); m/z 387 [M+H]⁺.

### b) 1-(4-fluoro-benzyl)-piperazine

### 8.2 (Z)-3-(4-Chloro-phenyl)-1-[4-(4-fluoro-benzyl)-piperazine-1-yl]-prop-2-en-1-one; compound B in Z-configuration

¹H NMR: δ(CDCl₃) 7.30 (m, 4H), 7.23 (m, 2H), 6-99 (dd, 2H), 6.61 (d, 1H), 6.05 (d, 1H), 3.67 (dd, 2H), 3.38 (s, 2H), 3.33 (dd, 2H), 238 (dd, 2H), 2.06 (dd, 2H).

### EXAMPLE 9

### (E)-(cis)-3-(4-Chloro-phenyl)-1-[4-(4-fluoro-benzyl)-2,5-dimethyl-piperazine-1-yl]-prop-2-en-1-one; compound E

Cis-2,5-Dimethylpiperazine dihydrobromide (248 mg, 0.9 mmol; T. T. Thang et al. J. Am. Chem. Soc. 1985, 50, 4913) in EtOH (10 mL) was treated with triethylamine (91 mg, 0.9 mmol). The mixture was heated at 60° C. and p-fluorobenzyl bromide (85 mg, 0.45 mmol) was added. After 30 min, a second portion of triethylamine (45 mg, 0.45 mmol) and p-fluorobenzyl bromide (42 mg, 0.22 mmol) were added to the reaction mixture. After an additional 1 h of stirring at 60° C., the last portion of triethylamine (46 mg, 0.45 mmol) and p-fluorobenzyl bromide (43 mg, 0-23 mmol) was added. The reaction mixture was allowed to attain room temperature and the solvent was removed in vacuo. The residue was dissolved in CH₂Cl₂, washed with aqueous 1 M NaOH and extracted with CH₂Cl₂. The organic layer was concentrated and submitted to flash column chromatography (EtOAc:MeOH:NEt₃ 30:2:1→10:1:1) to yield (4%) (cis)-1-(4-fluorobenzyl)-2,5-dimethyl-piperazine (8 mg, 0.036 mmol). (cis)-1-(4-fluorobenzyl)-2,5-dimethyl-piperazine (8 mg, 0.036 mmol) was reacted with (E)-p-chlorocinnamic acid in the same manner as described in Example 3 to give (E)-(cis)-3-(4-chloro-phenyl)-1-[4-(4-fluoro-benzyl)-2,5-dimethyl-piperazine-1-yl]-prop-2-en-1-one (10 mg, 0.025 mmol, yield: 72%).

Conformers in equilibrium, ¹H NMR: δ(CDCl₃) 7.62 (d, 1H), 7.45 (m, 2H), 7.34 (m, 2H), 7.30 (dd, 2H), 7.01 (dd, 2H), 6.81 (d, 1H), 4.77 (bs), 4.44 (d), 4.15 (m), 3.72 (d), 3.19 (m), 2.96 (d, 1H), 2.78 (m), 2.59 (d, 1H), 2.34 (bs, 1H), 2.17 (m, 1H), 1.29 (m, 3H), 1.23 (d, 3H).

Pharmacological Methods

In Vitro Assay

In the competitive affinity binding assay, the binding affinity of the compounds for the CCR1 receptor can be determined by measuring their ability to displace ¹²⁵I-Mip-1α from the CCR1 receptor.

The binding of Mip-1α at the CCR1 receptor leads to an increase of intracellular calcium levels. The ability of the compounds of the invention to block this biologic response of the CCR1 receptor is determined in the Ca²⁺-flux assay.

Since the binding of compounds to the CCR1 receptor need not to correlate with the biological activity of the receptor, the Ca²⁺-flux assay is more relevant to demonstrate the effect of the compounds of the invention.

In vitro Competitive Affinity Binding Assay

Reagents and Solutions:

1. Screen Ready™ Targets: cloned human CCR1 Chemokine receptor, expressed in CHO cells, coated on 96-well FlashPlate® (Perkin Elmer Cat #6120525)
2. Ligand: ¹²⁵I-MIP-1α from Perkin Elmer (specific activity is 2200 Ci/mmol) was reconstituted to 25 μCi/L in H₂O.
3. Assay buffer: 50 mM HEPES, 1 mM CaCl₂, 5 mM MgCl₂, 0.2% BSA, pH 7.4.
4. MIP-1α (Peprotech EC Ltd Cat # 300-08)
5. The compounds of the invention were dissolved in DMSO. A serial dilution was made and ten concentrations of each compound were screened to generate a dose curve from which the IC₅₀ value was determined.
   Assay Procedure:

Membranes coated on the FlashPlate® were incubated with ¹²⁵I-MIP-1α in the presence and absence of different concentrations of compounds at ambient temperature for 1 hour. The radioactivity in each well was determined in a microplate scintillation counter, The non-specific binding was defined by binding in the presence of 1250-fold unlabeled MIP-1α. The assay was performed according to the manufacturer's instruction of Screen Ready™ Targets. The compounds of the invention, when tested in this assay demonstrated affinity to the CCR1 receptor.

In vitro Ca²⁺-Flux Assay on Human Monocytes

Reagents and Solutions:

1. Cell Culture:
   a) THP-1 (ATCC Cat# TIB202)
   b) Tissue culture medium: RPMI 1640 with Ultraglutamine 1 supplemented with 10% (v/v) foetal calf serum. This medium is hereinafter referred to as “growth medium”.
2. Assay buffer: HMSS (Hanks' balanced salts solution), 20 mM HEPES, 1 mM CaCl₂, 1 mM MgCl₂, 2.5 mM Probenecid, pH 7.4.
3. Fluo-4AM (Molecular Probes Cat # F14201)
4. Pluronic® F-127 (Molecular Probes Cat # P-6867)
5. The compounds of the invention were dissolved in DMSO. A serial dilution was made and nine concentrations of each compound were screened to generate a dose curve from which the IC₅₀ value was determined.
6. MIP-1α (Peprotech EC Ltd Cat # 300-08)
7. Victor² 1420 (Perkin Elmer)
8. Microlite™ ²⁺ (Dynex Cat # 7572)
   Assay Procedure:

THP-1 cells were grown in T-75 cm² flasks in growth medium at 37° C. in 5% CO₂. The cells were harvested by centrifugation and resuspended in assay buffer. The cells were then loaded with 5 μM Fluo-4 and 0.02% pluronic acid (final concentrations) at 37° C. in 5% CO₂ for 30 min. The excess dye was removed by washing with assay buffer. The cells were resuspended and 10⁵ cells/well were added in a Microlite plate containing compounds and then incubated for 15 minutes at 37° C. in 5% CO₂. The cells were then stimulated with MIP-1α and changes in intracellular free Ca²⁺ concentration were measured with a Victor². The compounds of the invention, when tested in this assay, demonstrated the ability to inhibit the MIP-1α mediated Ca²⁺ mobilisation in THP-1 cells.

In Vivo Bioavailability in the Mouse

Female mice (SJL/N Tac) were given a single intravenous or oral dose of a mixture of 5 or 6 compounds per cassette (nominal dose: 1 mg/kg/compound) in a solution containing 0.5% N,N′-dimethylacetamide (DMA) and 15% sulfobutyl ether β-cyclodextrin (Captisol®). Blood samples were taken from one mouse per time point and dose group until 24 hour after respective administration. The dose formulations and plasma concentrations of each compound were determined by LC-MS/MS. The pharmacokinetic parameters were determined by non-compartmental analysis using WinNonlin Professional (version 4.0.1). The elimination rate constant, λ, was estimated by linear regression analysis of the terminal slope of the logarithmic plasma concentration-time curve. The area under the plasma concentration-time curve, AUC₀₋ₜ, was calculated by using the linear/logarithmic trapezoidal rule. The AUC_{inf} was calculated with the residual area estimated as C_{z}/λ. The calculated plasma concentration at the last time point, C_{z}, was obtained from the regression equation. The oral bioavailability (F) was calculated as:
Fₒᵣₐₗ=(AUC_{inf,po}/AUC_{inf,iv})·Doseᵢᵥ/Doseₚₒ).
Pharmacodynamic Assays

Using the procedures set forth in Horuk, R. and Ng, H. Med. Res. Rev. 2000, 20, 155 and Horuk, R. Methods, 2003, 29, 369 and references therein, the therapeutic efficacy of the compounds according to the invention for the treatment of inflammatory, autoimmune, proliferative or hyperproliferative diseases such as rheumatoid arthritis, multiple sclerosis, systemic lupus erythematosus, inflammatory bowel disease or asthma are shown.

Accordingly, in one embodiment of the invention a composition is provided comprising the compounds of formula 1 for the treatment of inflammatory, autoimmune, proliferative or hyperproliferative diseases.

The synergistic effect of combining the compounds according to the invention and cyclosporin A also is shown by use of methods mentioned in said references. Accordingly, in one embodiment of the invention a composition is provided comprising the compounds of formula I in combination with a sub-nephrotoxic amount of cyclosporin A. Using the procedures set forth in the competitive affinity binding assay and the Ca²⁺-flux assay, various compounds of the invention were tested for their affinity (IC₅₀^{af}) and ability to block Ca²⁺-flux (IC₅₀^{Ca}). The results of some examples and the Compounds A, B, C, D, E, and F (Compound D, E, and F are reference compounds) are shown in Table 3 where all IC₅₀-values are given in nM (nano Molar). Table 3 exemplifies the invention, without limiting the scope thereof.

TABLE 3

| Compound | Structure | IC₅₀^{af} (nM) | IC₅₀^{Ca} (nM) |
|---|---|---|---|
| A E-configuration Prior art | 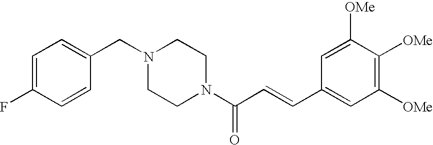 | 565 | 110 |
| 5.19 Invention | 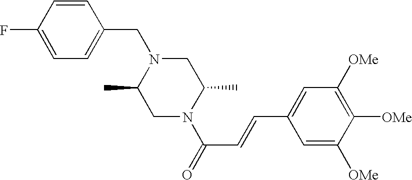 | 17 | 8 |
| 6.1 E-configuration Invention | 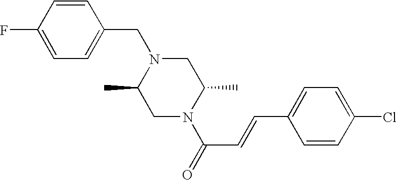 | 14 | 9 |
| 8.1; D Z-configuration Reference | 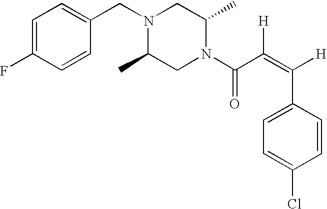 | >1000 | 207 |
| 9.1; E Reference | 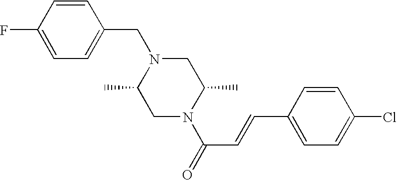 | | 910 |
| F Reference | 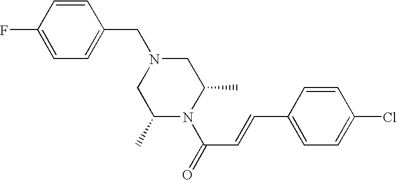 | >1000 | 416 |
| B E-configuration Prior art | 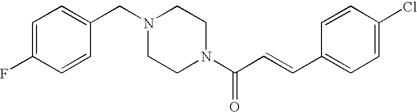 | 235 | 63 |
| 8.2; B Z-configuration Prior art | 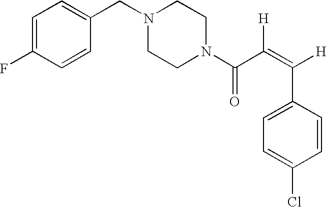 | >1000 | >1000 |
| 5.6 Invention | 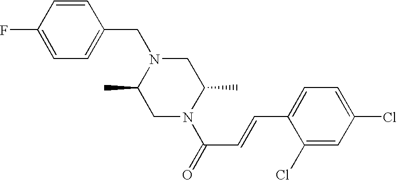 | | 18 |
| C Prior art | 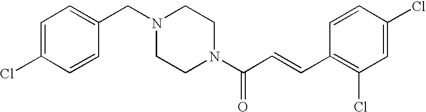 | | 120 |
| 6.2 Invention | 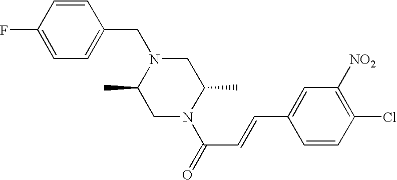 | 21 | 4 |
| 5.2 Invention | 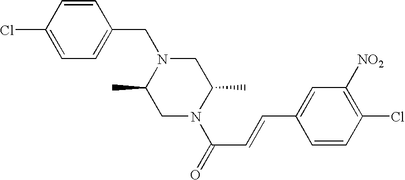 | 39 | 6 |
| 5.5 Invention | 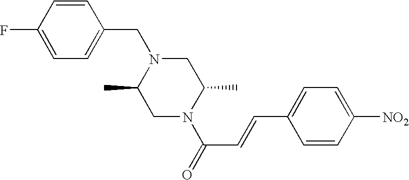 | 33 | 23 |
| 5.13 Invention | 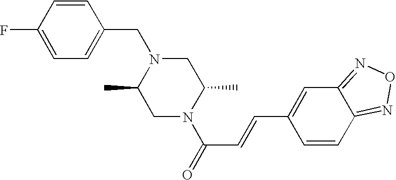 | 43 | 12 |
| 5.46 Invention | 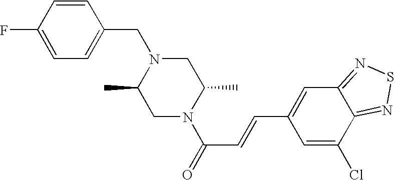 | | 7 |

Footnote:
All 2,5-dimethylpiperazine derivatives have been synthesized and tested as racemic mixtures.

The compounds of the invention show oral bioavailability in the mouse. Using the procedures set forth in the in vivo bioavailability assay, various compounds of the invention were tested for their clearance (CL; L/h/kg), plasma half-life (t_{1/2}; hrs) as well as oral bioavailability (F; %) after administration of the nominal dose of 1 mg/kg of each compound. The results of some examples are shown in Table 4. Table 4 exemplifies the invention, without limiting the scope thereof.

TABLE 4

| | | CL | t_{1/2} | F |
|---|---|---|---|---|
| Compound | Structure | (L/h/kg) | (hrs) | (%) |
| 6.1 | 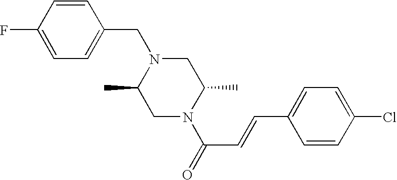 | 4.8 | 5.3 | 62 |
| 5.13 | 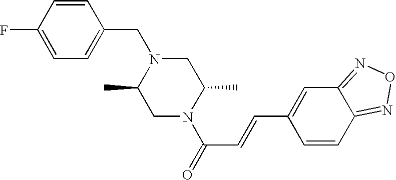 | 3.5 | 2.3 | 29 |

Administration

Effective quantities of the compounds of formula (I) are preferably administered to a patient in need of such treatment according to usual routes of administration and formulated in usual pharmaceutical compositions comprising an effective amount of the active ingredient and a suitable pharmaceutically acceptable carrier. Such compositions may take a variety of forms, e.g. solutions, suspensions, emulsions, tablets, capsules, and powders prepared for oral administration, sterile solutions for parental administration, suppositories for rectal administration or suitable topical formulations. Conventional procedures for the selection and preparation of suitable pharmaceutical formulations are described, for example, in Pharmaceuticals—The Science of Dosage Form Design, M. B. Aulton, Churchill Livingstone, 1988.

A suitable daily dose for use in the treatment of RA is contemplated to vary from 0.005 mg/kg to about 10 mg/kg body weight, in particular from 0.025 mg/kg to 2 mg/kg body weight, depending upon the specific condition to be treated, the age and weight of the specific patient, and the specific patient's response to the medication. The exact individual dosage, as well as the daily dosage, will be determined according to standard medical principles under the direction of a physician.

1. A compound of formula (I) 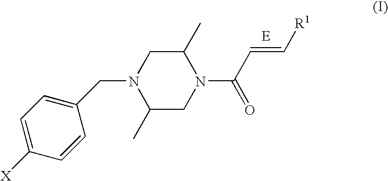
wherein:
the double bond in the amide moiety of formula (I) has an E-configuration;
X is a fluorine or a chlorine atom;
the methyl groups located at the 2- and 5-position of the piperazine ring are in trans-configuration to each other;
R¹ represents:
a) an aromatic group represented by the formula: 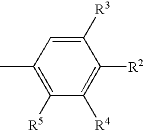
wherein:
R² is a substituent with a π-value between 0.5 and 0.9 and a mol refractory-value (MR) between 5.0 and 9.0, or R² is a nitro or methoxy substituent;
R³ is hydrogen, chloro, bromo, methyl, trifluoromethyl, methoxy or nitro, with the provisos that if R² is methoxy, R³ is methoxy, and if R² is nitro, R³ is hydrogen, chloro, methyl or trifluoromethyl;
R⁴ is hydrogen or methoxy, with the provisos that if R² is methoxy, R⁴ is selected from the group consisting of hydrogen, chloro, bromo and methoxy, or, if R³ is hydrogen, R⁴ is hydrogen;
R⁵ is hydrogen, chloro, or methyl, with the proviso that if R⁵ is chloro or methyl, X is fluoro, R² is chloro or methyl and R³ is hydrogen;
b) a heteroaromatic group represented by the formula: 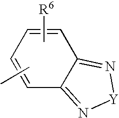
wherein:
Y is O or S;
R⁶ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
c) a heteroaromatic group represented by the formula: 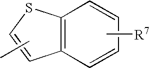
wherein:
R⁷ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
or a pharmaceutically acceptable salt or solvate thereof.

2. A compound according to claim 1 wherein:
R¹ represents:
a) an aromatic group represented by the formula: 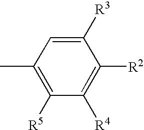
wherein:
R² is selected from the group consisting of methyl, chloro, bromo, trifluoromethyl, nitro and methoxy;
R³ is selected from the group consisting of hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from the group consisting of hydrogen and methoxy, with the provisos that if R² is methoxy, R⁴ is selected from a group consisting of hydrogen, chloro, or bromo or methoxy, or, if R³ is hydrogen, R⁴ is hydrogen;
R⁵ is hydrogen, chloro, methyl, with the proviso that if R⁵ is chloro or methyl, X is fluoro, R² is chloro or methyl and R³ is hydrogen;
b) a heteroaromatic group represented by the formula: 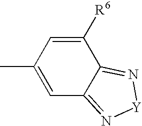
wherein:
R⁶ is one or more substituents independently selected from the group consisting of hydrogen, halo, methyl, ethyl, haloalkyl, alkoxy, haloalkoxy and nitro;
c) a heteroaromatic group represented by the formula: 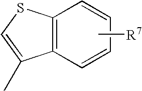
wherein:
R⁷ is one or more substituents independently selected from the group consisting of hydrogen, halo, C1-C3 alkyl, haloalkyl, alkoxy, haloalkoxy, nitro, cyano, alkylamino, aryl, alkylcarbonyl, and aminocarbonyl;
or a pharmaceutically acceptable salt or solvate thereof.

3. A compound according to claim 2 wherein X is fluorine.

4-23. (canceled)

24. A process for the preparation of a compound of formula (I) by 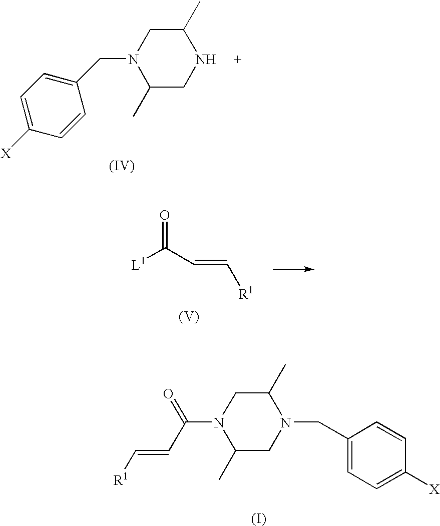
treating a piperazine derivative of formula (IV) with a compound of formula (V), wherein L¹ is a leaving group, in an organic solvent, at a temperature of 0° C. to 120° C.

25. A composition comprising a therapeutically effective amount of a compound of formula (I) 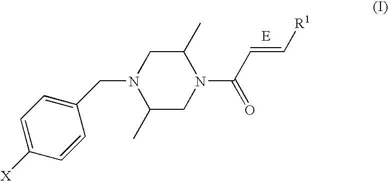
wherein:
the double bond in the amide moiety of formula (I) has an E-configuration;
X is a fluorine or a chlorine atom;
the methyl groups located at the 2- and 5-position of the piperazine ring are in trans-configuration to each other;
R¹ represents:
a) an aromatic group represented by the formula: 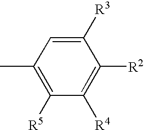
wherein:
R² is a substituent with a π-value between 0.5 and 0.9 and a mol refractory-value (MR) between 5.0 and 9.0, or R² is a nitro or methoxy substituent;
R³ is selected from the group consisting of hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from the group consisting of hydrogen and methoxy, with the provisos that if R² is methoxy, R⁴ is selected from the group consisting of hydrogen, chloro, bromo and methoxy, or, if R³ is hydrogen, R⁴ is hydrogen;
R⁵ is hydrogen, chloro, methyl, with the proviso that if R⁵ is chloro or methyl, X is fluoro, R² is chloro or methyl and R³ is hydrogen;
b) a heteroaromatic group represented by the formula: 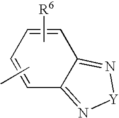
wherein:
Y is O or S;
R⁶ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
c) a heteroaromatic group represented by the formula: 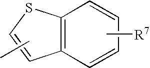
wherein:
R⁷ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
or a pharmaceutically acceptable salt or solvate thereof and pharmaceutically acceptable constituents, for use as a medicament.

26. Composition according to claim 25 further comprising a sub-nephrotoxic amount of cyclosporin A.

27. A method of treating a mammal suffering from inflammatory, autoimmune, proliferative or hyperproliferative disease, comprising administering to said mammal in need thereof a therapeutically effective amount of a compound formula (I) 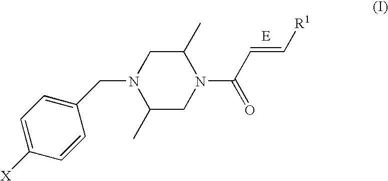
wherein:
the double bond in the amide moiety of formula (I) has an E-configuration;
X is a fluorine or a chlorine atom;
the methyl groups located at the 2- and 5-position of the piperazine ring are in trans-configuration to each other;
R¹ represents:
b) an aromatic group represented by the formula: 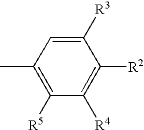
wherein:
R² is a substituent with a π-value between 0.5 and 0.9 and a mol refractory-value (MR) between 5.0 and 9.0, or R² is a nitro or methoxy substituent;
R³ is selected from the group consisting of hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from the group consisting of hydrogen and methoxy, with the provisos that if R² is methoxy, R⁴ is selected from the group consisting of hydrogen, chloro, bromo or methoxy, and, if R³ is hydrogen, R⁴ is hydrogen;
R⁵ is hydrogen, chloro, or methyl, with the proviso that if R⁵ is chloro or methyl, X is fluoro, R² is chloro or methyl and R³ is hydrogen;
b) a heteroaromatic group represented by the formula: 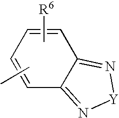
wherein:
Y is O or S;
R⁶ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
c) a heteroaromatic group represented by the formula: 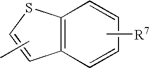
wherein:
R⁷ is one or more substituents independently selected from the group consisting of hydrogen, halo, C1-C4 alkyl, C2-C4 alkenyl, C2-C4 alkynyl, haloalkyl, alkoxy, haloalkoxy, hydroxyalkyl, hydroxy, alkylthio, alkylsulfonyl, alkylsulfinyl, nitro, cyano, alkylamino, aryl, amino, alkylsulfonylamino, dialkylsulfonamido, sulfonamido, carboxy, alkylcarbonyl, alkoxycarbonylalkyl, aminocarbonyl, monoalkylaminocarbonyl, dialkylaminocarbonyl, ureido and heteroaryl;
or a pharmaceutically acceptable salt or solvate thereof.

28. The method according to claim 27 wherein:
R¹ represents:
b) an aromatic group represented by the formula: 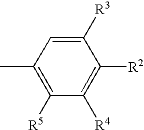
wherein:
R² is selected from the group consisting of methyl, chloro, bromo, trifluoromethyl, nitro and methoxy;
R³ is selected from the group consisting of hydrogen, chloro, bromo, methyl, trifluoromethyl, methoxy and nitro, with the provisos that if R² is methoxy, R³ is methoxy, and if R² is nitro, R³ is hydrogen, chloro, methyl or trifluoromethyl;
R⁴ is selected from the group consisting of hydrogen and methoxy, with the provisos that if R² is methoxy, R⁴ is selected from a group consisting of hydrogen, chloro, bromo or methoxy, or, if R is hydrogen, R⁴ is hydrogen;
R⁵ is hydrogen, chloro, or methyl, with the proviso that if R⁵ is chloro or methyl, X is fluoro, R² is chloro or methyl and R³ is hydrogen;
b) a heteroaromatic group represented by the formula: 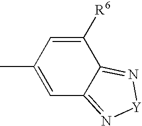
wherein:
R⁶ is one or more substituents independently selected from the group consisting of hydrogen, halo, methyl, ethyl, haloalkyl, alkoxy, haloalkoxy and nitro;
c) a heteroaromatic group represented by the formula: 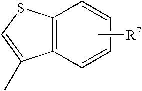
wherein:
R⁷ is one or more substituents independently selected from the group consisting of hydrogen, halo, C1-C3 alkyl, haloalkyl, alkoxy, haloalkoxy, nitro, cyano, alkylamino, aryl, alkylcarbonyl, and aminocarbonyl;
or a pharmaceutically acceptable salt or solvate thereof.

29. The method according to claim 28 wherein X is fluorine.

30-54. (canceled)

55. The method according to claim 27 of treating a mammal in need of treatment to prevent allograft rejection.

56. A compound according to claim 1 selected from the group consisting of
(E)-(trans)-3-(4-Bromo-phenyl)-1-[4-(4-fluoro-benzyl)-2,5-dimethyl-piperazine-1-yl]-prop-2-en-1-one;
(E)-(trans)-3-(4-Chloro-3-nitro-phenyl)-1-[4-(4-chloro-benzyl)-2,5-dimethyl-piperazine-1-yl]-prop-2-en-1-one;
(E)-(trans)-3-(3,4-Dichloro-phenyl)-1-[4-(4-fluoro-benzyl)-2,5-dimethyl-piperazine-1-yl]-prop-2-en-1-one;
(E)-(trans)-3-(2,4-Dichloro-phenyl)-1-[4-(4-fluoro-benzyl)-2,5-dimethyl-piperazine-1-yl]-prop-2-en-1-one hydrochloride;
(E)-(trans)-1-[4-(4-Chloro-benzyl)-2,5-dimethyl-piperazine-1-yl]-3-(4-chloro-phenyl)-prop-2-en-1-one;
(E)-(trans)-3-Benzo[2,1,3]thiadiazol-5-yl-1-[4-(4-fluoro-benzyl)-2,5-dimethyl-piperazine-1-yl]-prop-2-en-1-one;
(E)-(trans)-3-(4-Chloro-3-methoxy-5-nitro-phenyl)-1-[4-(4-fluoro-benzyl)-2,5-dimethyl-piperazine-1-yl]-prop-2-en-1-one;
(E)-(trans)-3-(4-Chloro-3-methoxy-phenyl)-1-[4-(4-fluoro-benzyl)-2,5-dimethyl-piperazine-1-yl]-prop-2-en-1-one;
(E)-(trans)-3-(4-Bromo-3,5-dimethoxy-phenyl)-1-[4-(4-fluorobenzyl)-2,5-dimethyl-piperazine-1-yl]-prop-2-en-1-one;
(E)-(trans)-1-[4-(4-Fluorobenzyl)-2,5-dimethyl-piperazine-1-yl]-3-(3-methoxy-4-methyl-phenyl)-prop-2-en-1-one;
(E)-(trans)-3-(4-Bromo-phenyl)-1-[4-(4-chlorobenzyl)-2,5-dimethyl-piperazine-1-yl]-prop-2-en-1-one;
(E)-(trans)-3-(3-Bromo-4-chloro-phenyl)-1-[4-(4-fluorobenzyl)-2,5-dimethyl-piperazine-1-yl]-prop-2-en-1-one;
(E)-(trans)-3-(4-Bromo-3-chloro-phenyl)-1-[4-(4-fluorobenzyl)-2,5-dimethyl-piperazine-1-yl]-prop-2-en-1-one;
(E)-(trans)-3-(3,4-Dibromo-phenyl)-1-[4-(4-fluorobenzyl)-2,5-dimethyl-piperazine-1-yl]-prop-2-en-1-one;
(E)-(trans)-3-(4-Bromo-3-nitro-phenyl)-1-[4-(4-fluorobenzyl)-2,5-dimethyl-piperazine-1-yl]-prop-2-en-1-one;
(E)-(trans)-3-(4-Bromo-benzo[2,1,3]thiadiazol-6-yl)-1-[4-(4-fluorobenzyl)-2,5-dimethyl-piperazine-1-yl]-prop-2-en-1-one;
(E)-(trans)-3-(4-Chloro-benzo[2,1,3]thiadiazol-6-yl)-1-[4-(4-fluorobenzyl)-2,5-dimethyl-piperazine-1-yl]-prop-2-en-1-one;
(E)-(trans)-1-[4-(4-Fluoro-benzyl)-2,5-dimethyl-piperazine-1-yl]-3-(4-nitro-benzo[2,1,3]thiadiazol-5-yl)-prop-2-en-1-one;
(E)-(trans)-3-(4-Chloro-phenyl)-1-[4-(4-fluoro-benzyl)-2,5-dimethyl-piperazine-1-yl]-prop-2-en-1-one; and
(E)-(trans)-3-(4-Chloro-3-nitro-phenyl)-1-[4-(4-fluoro-benzyl)-2,5-dimethyl-piperazine-1-yl]-prop-2-en-1-one.

57. The method according to claim 27 wherein the compound as administered is selected from the group consisting of
(E)-(trans)-3-(4-Bromo-phenyl)-1-[4-(4-fluoro-benzyl)-2,5-dimethyl-piperazine-1-yl]-prop-2-en-1-one;
(E)-(trans)-3-(4-Chloro-3-nitro-phenyl)-1-[4-(4-chloro-benzyl)-2,5-dimethyl-piperazine-1-yl]-prop-2-en-1-one;
(E)-(trans)-3-(3,4-Dichloro-phenyl)-1-[4-(4-fluoro-benzyl)-2,5-dimethyl-piperazine-1-yl]-prop-2-en-1-one;
(E)-(trans)-3-(2,4-Dichloro-phenyl)-1-[4-(4-fluoro-benzyl)-2,5-dimethyl-piperazine-1-yl]-prop-2-en-1-one hydrochloride;
(E)-(trans)-1-[4-(4-Chloro-benzyl)-2,5-dimethyl-piperazine-1-yl]-3-(4-chloro-phenyl)-prop-2-en-1-one;
(E)-(trans)-3-Benzo[2,1,3]thiadiazol-5-yl-1-[4-(4-fluoro-benzyl)-2,5-dimethyl-piperazine-1-yl]-prop-2-en-1-one;
(E)-(trans)-3-(4-Chloro-3-methoxy-5-nitro-phenyl)-1-[4-(4-fluoro-benzyl)-2,5-dimethyl-piperazine-1-yl]-prop-2-en-1-one;
(E)-(trans)-3-(4-Chloro-3-methoxy-phenyl)-1-[4-(4-fluoro-benzyl)-2,5-dimethyl-piperazine-1-yl]-prop-2-en-1-one;
(E)-(trans)-3-(4-Bromo-3,5-dimethoxy-phenyl)-1-[4-(4-fluorobenzyl)-2,5-dimethyl-piperazine-1-yl]-prop-2-en-1-one;
(E)-(trans)-1-[4-(4-Fluorobenzyl)-2,5-dimethyl-piperazine-1-yl]-3-(3-methoxy-4-methyl-phenyl)-prop-2-en-1-one;
(E)-(trans)-3-(4-Bromo-phenyl)-1-[4-(4-chlorobenzyl)-2,5-dimethyl-piperazine-1-yl]-prop-2-en-1-one;
(E)-(trans)-3-(3-Bromo-4-chloro-phenyl)-1-[4-(4-fluorobenzyl)-2,5-dimethyl-piperazine-1-yl]-prop-2-en-1-one;
(E)-(trans)-3-(4-Bromo-3-chloro-phenyl)-1-[4-(4-fluorobenzyl)-2,5-dimethyl-piperazine-1-yl]-prop-2-en-1-one;
(E)-(trans)-3-(3,4-Dibromo-phenyl)-1-[4-(4-fluorobenzyl)-2,5-dimethyl-piperazine-1-yl]-prop-2-en-1-one;
(E)-(trans)-3-(4-Bromo-3-nitro-phenyl)-1-[4-(4-fluorobenzyl)-2,5-dimethyl-piperazine-1-yl]-prop-2-en-1-one;
(E)-(trans)-3-(4-Bromo-benzo[2,1,3]thiadiazol-6-yl)-1-[4-(4-fluorobenzyl)-2,5-dimethyl-piperazine-1-yl]-prop-2-en-1-one;
(E)-(trans)-3-(4-Chloro-benzo[2,1,3]thiadiazol-6-yl)-1-[4-(4-fluorobenzyl)-2,5-dimethyl-piperazine-1-yl]-prop-2-en-1-one;
(E)-(trans)-1-[4-(4-Fluoro-benzyl)-2,5-dimethyl-piperazine-1-yl]-3-(4-nitro-benzo[2,1,3]thiadiazol-5-yl)-prop-2-en-1-one;
(E)-(trans)-3-(4-Chloro-phenyl)-1-[4-(4-fluoro-benzyl)-2,5-dimethyl-piperazine-1-yl]-prop-2-en-1-one; and
(E)-(trans)-3-(4-Chloro-3-nitro-phenyl)-1-[4-(4-fluoro-benzyl)-2,5-dimethyl-piperazine-1-yl]-prop-2-en-1-one.

58. The method according to claim 27 of treating a mammal suffering from rheumatoid arthritis; multiple sclerosis; systemic lupus erythematosus; inflammatory bowel disease or asthma.
